(19) United States
(12) Patent Application Publication
(10) Pub. No.: US 2026/0011440 A1
(43) Pub. Date: Jan. 8, 2026

(54) COMMUNICATION SYSTEM FOR PATIENT SUPPORT APPARATUSES AND OTHER EQUIPMENT

(71) Applicant: Stryker Corporation, Portage, MI (US)

(72) Inventors: Celso Henrique Farnese Pires Pereira, Portage, MI (US); Kirby M. Neihouser, Portage, MI (US); Madhu Sandeep Thota, Portage, MI (US); Jerald A. Trepanier, Augusta, MI (US); Krishna Sandeep Bhimavarapu, Kalamazoo, MI (US)

(21) Appl. No.: 19/132,923

(22) PCT Filed: Nov. 27, 2023
(86) PCT No.: PCT/US2023/081072

§ 371 Date: May 27, 2025

Related U.S. Application Data
(60) Provisional application No. 63/428,076, filed on Nov. 27, 2022.

(51) Int. Cl. G16H 40/67; A61F 7/00; G01S 13/02; G16H 40/20; G16H 40/63

(52) U.S. Cl. CPC G16H 40/67; A61F 7/0053; G01S 13/0209; G16H 40/20; G16H 40/63

(57) ABSTRACT

A system for a healthcare facility includes a server and a device comprising a UWB transceiver, a controller, and a network transceiver for communicating with the server. The server is adapted to equate a location of the device with a location of a patient support apparatus location if the device is positioned within a first distance of the patient support apparatus, and to equate the location of the device with a location of a fixed locator if the device is not positioned within the first distance of the patient support apparatus, and the device is positioned within a second distance of the fixed locator. The controller may be adapted to automatically attempt to pair with a patient support apparatus and, if it is unable to pair with the patient support apparatus, to automatically attempt to pair with a fixed locator mounted at a fixed location within a healthcare facility.

### BACKGROUND

The present disclosure relates to medical equipment that is used during the course of a patient's stay at a medical facility. More specifically, the present disclosure relates to medical equipment that communicates with other devices within a patient's room and/or with a remote server.

### SUMMARY

According to the various aspects described herein, the present disclosure is directed to a system that monitors the associations of medical equipment within a healthcare facility. Such associations may be associations that correlate medical equipment to specific patients, to specific caregivers, to specific locations, to specific electronic medical records, to other pieces of equipment, and/or to other parameters. The system may automatically determine what locations and/or other parameters various medical equipment should be associated with, and distribute this information to healthcare personnel and/or other systems within the healthcare facility so that caregivers do not have to manually locate equipment and/or make manual associations of the equipment to other equipment, patients, caregivers, and/or other parameters. The system of the present disclosure may also or additionally automatically pair one or more pieces of equipment with each other so that communications between the pieces of equipment may be automatically established and disestablished based on the proximity of the pieces of equipment to each other and/or to other structures. The automatic establishment of communications enables the equipment to share information not only with each other, but also to utilize each other as communications conduits for communicating with the server and/or other systems that are part of the healthcare facility's computer network. These and other aspects of the present disclosure will be apparent to a person of ordinary skill in the art in light of the following written description and accompanying drawings.

According to a first aspect of the present disclosure, a device is provided that includes an ultra-wideband transceiver and a controller. The controller is adapted to use the ultra-wideband transceiver to attempt to determine a first distance between the ultra-wideband transceiver and a patient support apparatus. If the controller is unable to determine the first distance between the ultra-wideband transceiver and the patient support apparatus, or if the first distance is greater than a first threshold, the controller is further adapted to use the ultra-wideband transceiver to attempt to determine a second distance between the ultra-wideband transceiver and a fixed locator mounted at a fixed location within a healthcare facility.

According to other aspects of the present disclosure, if the second distance is less than a second threshold, the controller is further adapted to associate the device with the fixed locator.

In some aspects, the device further includes a network transceiver adapted to communicate with a network of the healthcare facility. The controller may be further adapted to send a fixed locator ID to the network if the second distance is less than the second threshold, and to not send the fixed locator ID to the network if the second distance is greater than the second threshold.

In some aspects, the controller and ultra-wideband transceiver are adapted to act as a UWB tag when the controller is unable to determine the first distance or when the first distance is greater than the first threshold. The controller and ultra-wideband transceiver are further adapted to act as a UWB anchor when the distance is less than the first threshold.

In some aspects, when the controller and ultra-wideband transceiver are acting as a UWB tag, the ultra-wideband transceiver is adapted to periodically transmit a discovery packet (e.g. a start packet) to any other ultra-wideband transceivers within range and to wait for a response from the any other ultra-wideband transceivers.

When the controller and ultra-wideband transceiver are acting as a UWB anchor, in some aspects, the controller and ultra-wideband transceiver are adapted to respond to a discovery packet transmitted by another ultra-wideband transceiver.

When the controller and ultra-wideband transceiver are acting as a UWB tag, in some aspects, the ultra-wideband transceiver and controller are adapted to ignore a discovery packet transmitted by another ultra-wideband transceiver.

When the controller and ultra-wideband transceiver are acting as a UWB anchor, in some aspects, the ultra-wideband transceiver is adapted to not transmit any discovery packets to another ultra-wideband transceiver.

In some aspects, the device is a patient temperature management unit adapted to control a patient's temperature during a thermal therapy session.

In some aspects, the thermal control unit includes a first fluid port adapted to fluidly couple to a first hose; a second fluid port adapted to fluidly couple to a second hose; a fluid channel fluidly coupling the first fluid port to the second fluid port; a pump for pumping fluid through the thermal control unit; a heat exchanger adapted to add or remove heat from the fluid; a fluid temperature sensor adapted to sense a temperature of the fluid; a patient temperature sensor port adapted to receive patient temperature readings from a patient temperature sensor; and a temperature controller adapted to control the heat exchanger in order to control the patient's temperature.

In some aspects, the controller is further adapted to receive a fixed locator ID from the fixed locator if the second distance is less than a second threshold, and the controller is further adapted to transmit the fixed locator ID using the ultra-wideband transceiver to another device if the another device is within a third threshold.

If the controller associates the device with the fixed locator, in some aspects, the controller is further adapted to repetitively attempt to determine the first distance between the ultra-wideband transceiver and the patient support apparatus.

In some aspects, if the controller is able to determine the first distance between the ultra-wideband transceiver and the patient support apparatus, and the first distance is less than the first threshold, the controller is adapted to automatically disassociate the device from the fixed locator and associate the device with the patient support apparatus.

The controller, in some aspects, is further adapted to determine, after the device is associated with the fixed locator, if another device is within a third threshold of the device, and if the another device is within the third threshold of the device, to transmit a fixed locator ID received from the fixed locator to the another device.

In some aspects, if the another device is not within the third threshold of the device, the controller is adapted to prevent the transmission of the fixed locator ID to the another device.

The device, in some aspects, is a medical device adapted to be used in the treatment of a patient in the healthcare facility.

The device, in some aspects, is one of the following: an infusion pump, a vital sign sensor, an exercise device, a heel care boot, an IV stand and/or pole, a ventilator, a Deep Vein Thrombosis treatment device, a patient monitor, a patient positioning devices, a temperature sensor, a mattress, a portable exit detection sensor, an attachable nurse call device, an incontinence detector, a Holter device adapted to monitor and record a patient's heart signals, a patient ID tag adapted to be worn by a patient, a caregiver ID tag adapted to be worn by a caregiver, or a mobility device adapted to assist movement of the patient.

In some aspects, the first distance is any distance within a first three-dimensional space defined around the patient support apparatus, and the second distance is any distance within a second three-dimensional space defined around the fixed locator.

According to another aspects of the present disclosure, a device is provided that includes an ultra-wideband transceiver and a controller. The controller is adapted to use the ultra-wideband transceiver to automatically attempt to pair with a patient support apparatus and, if the controller is unable to pair with the patient support apparatus, the controller is further adapted to use the ultra-wideband transceiver to automatically attempt to pair with a fixed locator mounted at a fixed location within a healthcare facility.

In some aspects, the controller is further adapted to not attempt to pair with the fixed locator if the controller is able to pair with the patient support apparatus.

The controller, in some aspects, is further adapted, after pairing with the fixed locator, to automatically re-attempt to pair with the patient support apparatus.

In some aspects, the controller is adapted to automatically unpair the device from the fixed locator if the controller is able to pair with the patient support apparatus.

The controller, in some aspects, is adapted to accept a fixed locator ID from the fixed locator when the device is paired with the fixed locator.

The device, in some aspects, further includes a network transceiver adapted to communicate with a network of the healthcare facility, and the controller is further adapted to send the fixed locator ID to the network.

The controller, in some aspects, is further adapted to send the fixed locator ID to another device if the another device is within a threshold distance of the device.

The controller, in some aspects, is configured to attempt to pair with the patient support apparatus by determining a first distance between the ultra-wideband transceiver and a second ultra-wideband transceiver on the patient support apparatus.

In some aspects, the first distance is any distance within a three-dimensional space defined around the patient support apparatus.

The controller, in some aspects, is configured to attempt to pair with the fixed locator by determining a second distance between the ultra-wideband transceiver and a third ultra-wideband transceiver on the fixed locator.

The second distance, in some aspects, is any distance within a three-dimensional space defined around the fixed locator.

In some aspects, the controller and ultra-wideband transceiver are adapted to act as a UWB tag when the controller is attempting to pair with the patient support apparatus and to act as a UWB anchor when attempting to pair with the fixed locator.

When acting as a UWB tag, in some aspects, the ultra-wideband transceiver is adapted to periodically transmit a discovery packet to any other ultra-wideband transceivers within range and to wait for a response from the any other ultra-wideband transceivers.

When acting as a UWB anchor, in some aspects, the controller and ultra-wideband transceiver are adapted to respond to a discovery packet transmitted by another ultra-wideband transceiver.

When acting as a UWB tag, in some aspects, the ultra-wideband transceiver and controller are adapted to ignore a discovery packet transmitted by another ultra-wideband transceiver.

When acting as a UWB anchor, in some aspects, the ultra-wideband transceiver is adapted to not transmit any discovery packets to another ultra-wideband transceiver.

In some aspects, the device is a thermal control unit adapted to control a patient's temperature during a thermal therapy session.

In some aspects, the thermal control unit includes a first fluid port adapted to fluidly couple to a first hose; a second fluid port adapted to fluidly couple to a second hose; a fluid channel fluidly coupling the first fluid port to the second fluid port; a pump for pumping fluid through the thermal control unit; a heat exchanger adapted to add or remove heat from the fluid; a fluid temperature sensor adapted to sense a temperature of the fluid; a patient temperature sensor port adapted to receive patient temperature readings from a patient temperature sensor; and a temperature controller adapted to control the heat exchanger in order to control the patient's temperature.

The device, in some aspects, is a medical device adapted to be used in the treatment of a patient in the healthcare facility.

The device, in some aspects, is one of the following: an infusion pump, a vital sign sensor, an exercise device, a heel care boot, an IV stand and/or pole, a ventilator, a Deep Vein Thrombosis treatment device, a patient monitor, a patient positioning devices, a temperature sensor, a mattress, a portable exit detection sensor, an attachable nurse call device, an incontinence detector, a Holter device adapted to monitor and record a patient's heart signals, a patient ID tag adapted to be worn by a patient, a caregiver ID tag adapted to be worn by a caregiver, or a mobility device adapted to assist movement of the patient.

According to another aspect of the present disclosure, a system is provided that includes a server and a device. The server is adapted to be accessible from a network of a healthcare facility. The device includes a first UWB transceiver, a controller, and a network transceiver adapted to be able to communicate with the server. The server is further adapted to equate a location of the device with a location of a patient support apparatus if the device is positioned within a first distance of the patient support apparatus, and to equate the location of the device with a location of a fixed locator if both of the following are true: (1) the device is not positioned within the first distance of the patient support apparatus, and (2) the device is positioned within a second distance of the fixed locator.

In some aspects, the first and second distances are the same, while in other aspects, the first and second distances may be different.

In some aspects, the server is adapted to receive a fixed locator ID from the device if the device is not positioned within the first distance of the patient support apparatus and the device is positioned within the second distance of the fixed locator.

The server, in some aspects, is adapted to receive the fixed locator ID from the patient support apparatus if the device is positioned within the first distance of the patient support apparatus.

The fixed locator, in some aspects, is adapted to transmit a fixed locator ID to the patient support apparatus if the patient support apparatus if positioned within a third distance of the fixed locator, to not transmit the fixed locator ID to the patient support apparatus if the patient support apparatus is positioned outside of the third distance from the fixed locator, to transmit the fixed locator ID to the device if the device is positioned within the second distance of the fixed locator, and to not transmit the fixed locator ID to the device if the device is positioned outside of the second distance from the fixed locator.

The server, in some aspects, is adapted to receive data from the device after the data passes through the patient support apparatus if the device is positioned within the first distance of the patient support apparatus, and to receive data from the device without the data passing through the patient support apparatus if both of the following are true: (1) the device is not positioned within the first distance of the patient support apparatus, and (2) the device is positioned within the second distance of the fixed locator.

The server, in some aspects, is adapted to communicate with a display, and the server may be adapted to instruct the display to display the location of the device in the same manner regardless of whether the device is positioned within the first distance of the patient support apparatus or the device is positioned within the second distance of the fixed locator.

In some aspects, the controller is adapted to use the ultra-wideband transceiver to automatically attempt to pair with the patient support apparatus, and if the controller is unable to pair with the patient support apparatus, the controller is further adapted to use the ultra-wideband transceiver to automatically attempt to pair with the fixed locator mounted.

The controller, in some aspects, is further adapted to not attempt to pair with the fixed locator if the controller is able to pair with the patient support apparatus.

In some aspects, the controller is further adapted, after pairing with the fixed locator, to automatically re-attempt to pair with the patient support apparatus.

The controller, in some aspects, is adapted to automatically unpair the device from the fixed locator if the controller is able to pair with the patient support apparatus.

The controller, in some aspects, is adapted to accept a fixed locator ID from the fixed locator when the device is paired with the fixed locator.

The controller, in some aspects, is further adapted to send the fixed locator ID to the network using the network transceiver.

In some aspects, the controller is further adapted to send the fixed locator ID to another device if the another device is within a threshold distance of the device.

The first distance, in some aspects, is any distance within a three-dimensional space defined around the patient support apparatus.

The second distance, in some aspects, is any distance within a three-dimensional space defined around the fixed locator.

In some aspects, the three-dimensional space for either or both of the first and second distances may define a volume of space that is non-symmetrical along at least one axis.

The controller and UWB transceiver, in some aspects, are adapted to act as a UWB tag when the controller is attempting to pair with the patient support apparatus and to act as a UWB anchor when attempting to pair with the fixed locator.

When acting as a UWB tag, in some aspects, the ultra-wideband transceiver is adapted to periodically transmit a discovery packet to any other ultra-wideband transceivers within range and to wait for a response from the any other ultra-wideband transceivers.

When acting as a UWB anchor, in some aspects, the controller and ultra-wideband transceiver are adapted to respond to a discovery packet transmitted by another ultra-wideband transceiver.

When acting as a UWB tag, in some aspects, the ultra-wideband transceiver and controller are adapted to ignore a discovery packet transmitted by another ultra-wideband transceiver.

When acting as a UWB anchor, in some aspects, the ultra-wideband transceiver is adapted to not transmit any discovery packets to another ultra-wideband transceiver.

In some aspects, the device is a thermal control unit adapted to control a patient's temperature during a thermal therapy session.

In some aspects, the thermal control unit includes a first fluid port adapted to fluidly couple to a first hose; a second fluid port adapted to fluidly couple to a second hose; a fluid channel fluidly coupling the first fluid port to the second fluid port; a pump for pumping fluid through the thermal control unit; a heat exchanger adapted to add or remove heat from the fluid; a fluid temperature sensor adapted to sense a temperature of the fluid; a patient temperature sensor port adapted to receive patient temperature readings from a patient temperature sensor; and a temperature controller adapted to control the heat exchanger in order to control the patient's temperature.

The device, in some aspects, is a medical device adapted to be used in the treatment of a patient in the healthcare facility.

The device, in some aspects, is one of the following: an infusion pump, a vital sign sensor, an exercise device, a heel care boot, an IV stand and/or pole, a ventilator, a Deep Vein Thrombosis treatment device, a patient monitor, a patient positioning devices, a temperature sensor, a mattress, a portable exit detection sensor, an attachable nurse call device, an incontinence detector, a Holter device adapted to monitor and record a patient's heart signals, a patient ID tag adapted to be worn by a patient, a caregiver ID tag adapted to be worn by a caregiver, or a mobility device adapted to assist movement of the patient.

In some aspects, the controller is adapted to use radio frequency (RF) communication between the ultra-wideband transceiver and a badge worn by a user to determine a position of the badge relative to the device; to receive a badge identifier from the badge; to determine if the badge is positioned inside or outside of a threshold distance; and to transmit the badge identifier to the server using the network transceiver if the badge is inside the threshold distance.

In some aspects, the server is adapted to use the badge identifier to determine an identity of the user associated with the badge.

Before the various aspects of the disclosure are explained in detail, it is to be understood that the claims are not to be limited to the details of operation or to the details of construction and the arrangement of the components set forth in the following description or illustrated in the drawings. The aspects described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus and a patient temperature management device according to a first aspect of the present disclosure;

FIG. 2 is a plan view of an illustrative caregiver control panel of the patient support apparatus of FIG. 1;

FIG. 3 is a plan view of an illustrative patient control panel of the patient support apparatus of FIG. 1;

FIG. 4 is a perspective view of the patient temperature management device of FIG. 1;

FIG. 5 is a block diagram of the patient temperature management device;

FIG. 6 is a diagram of the patient support apparatus, the patient temperature management device, a display device, a fixed locator unit, and several components of a healthcare facility local area network;

FIG. 7 is a block diagram of the patient support apparatus, the patient temperature management device, the fixed locator unit, and the display;

FIG. 8 is a block diagram of the temperature management device, the fixed locator unit, and the display;

FIG. 9 is a flow diagram of a communication and association algorithm followed by a primary UWB device, such as a patient support apparatus

FIG. 10 is a flow diagram of a communication and association algorithm followed by a secondary UWB device, such as, but not limited to, the temperature management device;

FIG. 11 is a flow diagram of a communication and association algorithm followed by a tertiary UWB device; and

FIG. 12 is an example of a dashboard screen that may be displayed on an electronic device in communication with a server on the healthcare facility's local network.

### DETAILED DESCRIPTION OF THE EMBODIMENTS

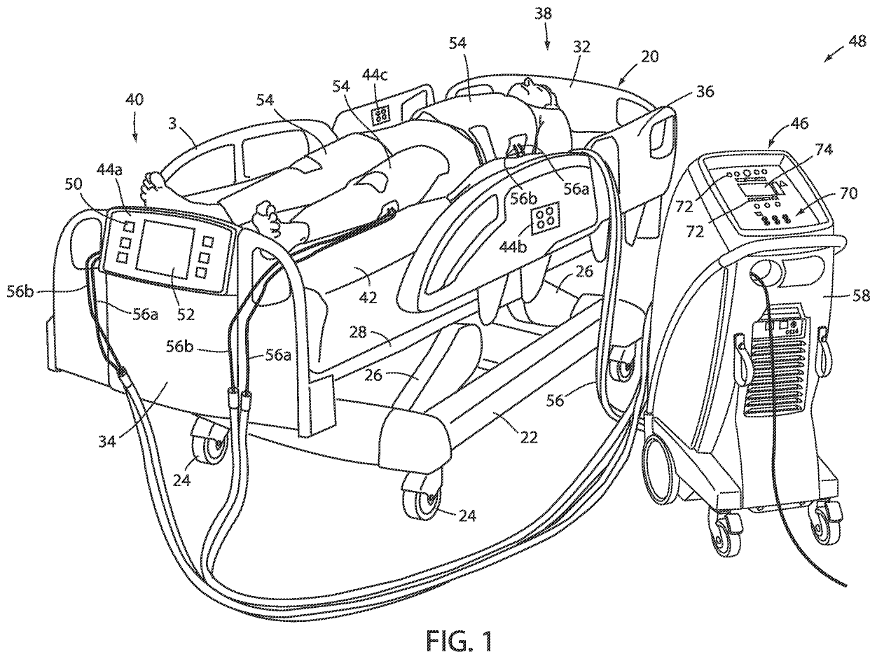

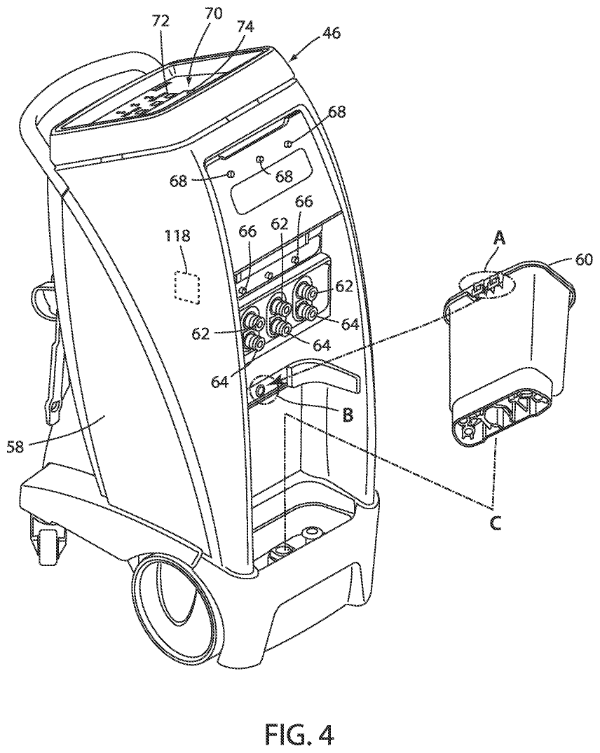

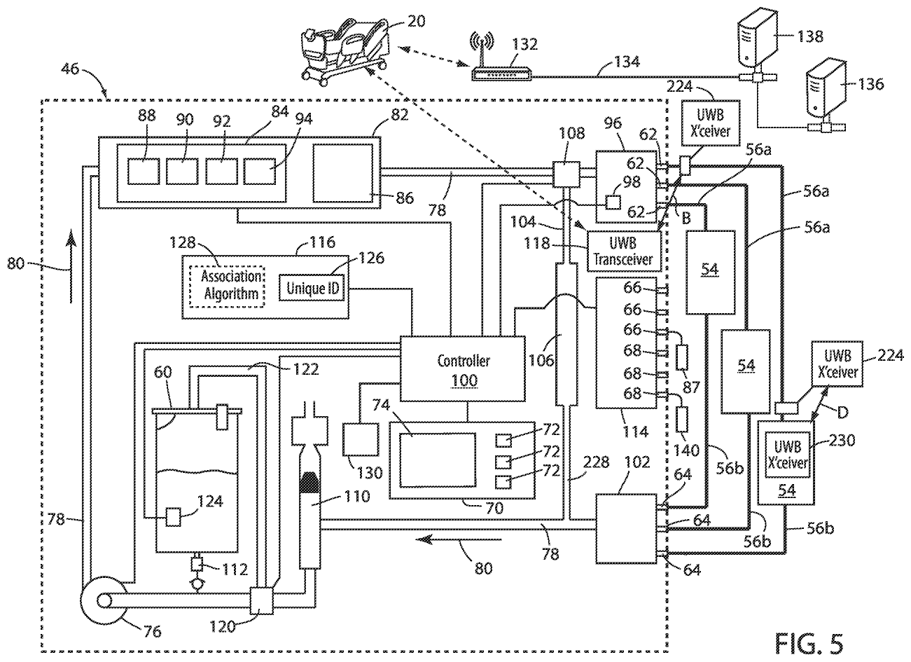

An illustrative patient support apparatus 20 and a thermal control system 48 according to several aspects of the present disclosure are shown in FIG. 1. The thermal control system 48 is used to control the temperature of a patient while he or she is positioned on patient support apparatus 20, and further details of this system 48 are provided below in the discussion of FIGS. 4-5. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, an operating table, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. In some embodiments, the mattress 42 includes one or more inflatable bladders that are controllable via a blower, or other source of pressurized air. In at least one embodiment, the inflation of the bladders of the mattress 42 is controllable via electronics built into patient support apparatus 20. In one such embodiments, mattress 42 may take on any of the functions and/or structures of any of the mattresses disclosed in commonly assigned U.S. Pat. No. 9,468,307 issued Oct. 18, 2016, to inventors Patrick Lafleche et al., the complete disclosure of which is incorporated herein by reference. Still other types of mattresses may be used.

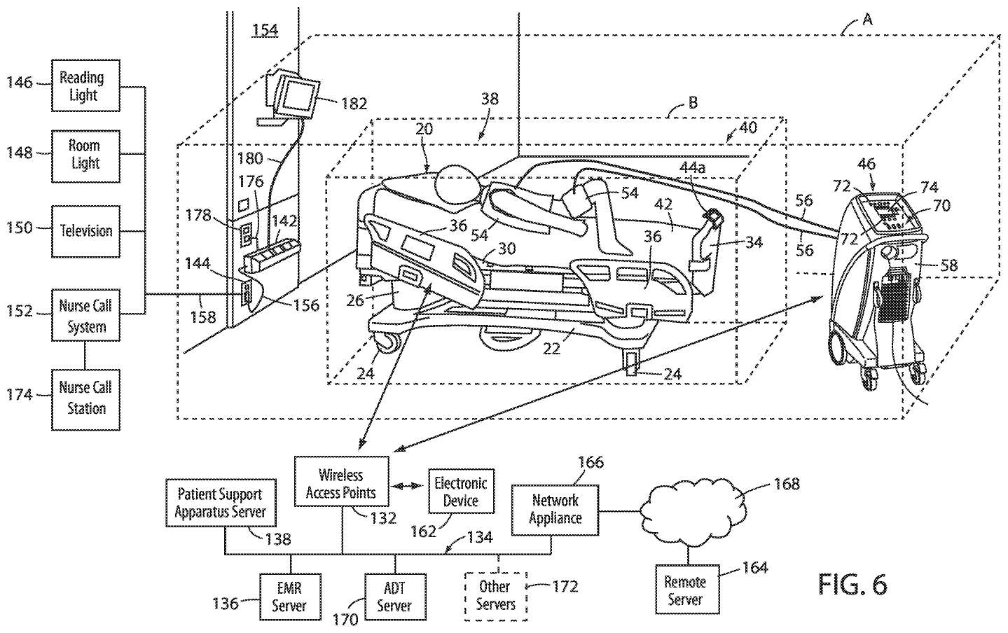

Support deck 30 is made of a plurality of sections, some of which are pivotable about generally horizontal pivot axes. In the embodiment shown in FIG. 1, support deck 30 includes at least a head section, a thigh section, and a foot section, all of which are positioned underneath mattress 42 and which generally form flat surfaces for supporting mattress 42. The head section, which is also sometimes referred to as a Fowler section, is pivotable about a generally horizontal pivot axis between a generally horizontal orientation (shown in FIG. 1) and a plurality of raised positions (one of which is shown in FIG. 6). The thigh section and foot section may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width.

It will be understood by those skilled in the art that patient support apparatus 20 can be designed with other types of mechanical constructions that are different from what is shown in the attached drawings, such as, but not limited to, the construction described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may include the same, or nearly the same, structures as the Model 3002 S3 bed manufactured and sold by Stryker Corporation of Kalamazoo, Michigan. This construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. In still another embodiment, the mechanical construction of patient support apparatus 20 may include the same, or nearly the same, structure as the Model 3009 Procuity MedSurg bed manufactured and sold by Stryker Corporation of Kalamazoo, Michigan. This construction is described in greater detail in the Stryker Maintenance Manual for the 3009 Procuity MedSurg bed (publication 3009-009-002, Rev. A.0), published in 2020 by Stryker Corporation of Kalamazoo, Michigan.

It will be understood by those skilled in the art that patient support apparatus 20 can be designed with still other types of mechanical constructions, such as, but not limited to, those described in commonly assigned, U.S. Pat. No. 7,690,059 issued Apr. 6, 2010, to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The overall mechanical construction of patient support apparatus 20 may also take on still other forms different from what is disclosed in the aforementioned references provided the patient support apparatus includes one or more of the functions, features, and/or structures discussed in greater detail below.

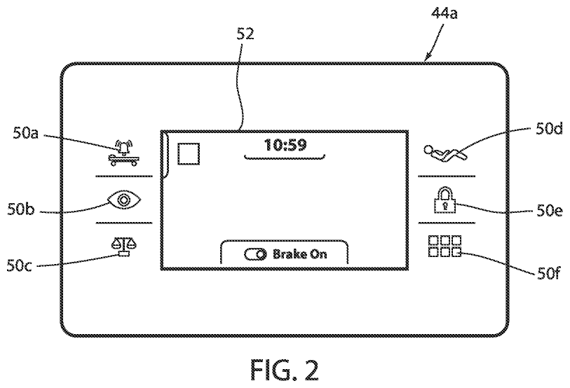

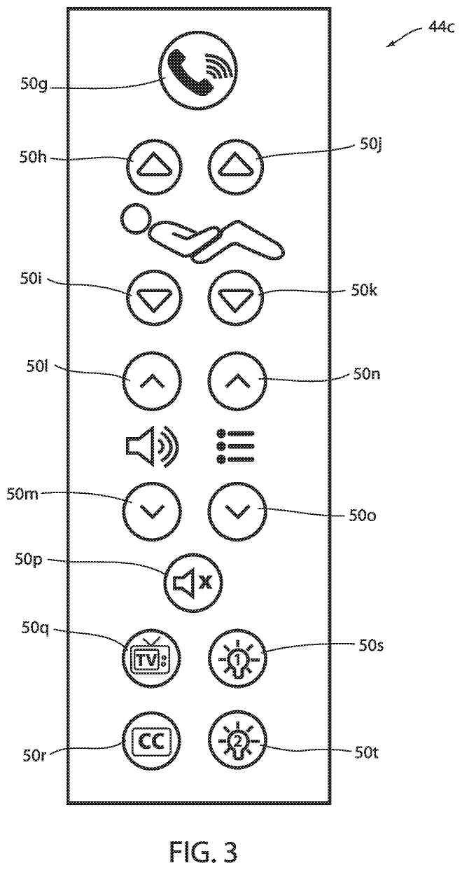

Patient support apparatus 20 further includes a plurality of control panels 44 that enable a user of patient support apparatus 20, such as a patient and/or an associated caregiver, to control one or more aspects of patient support apparatus 20. In the embodiment shown in FIG. 1, patient support apparatus 20 includes a footboard control panel 44a, a pair of outer siderail control panels 44b (only one of which is visible), and a pair of inner siderail control panels 44c (only one of which is visible). Footboard control panel 44a and outer siderail control panels 44b are intended to be used by caregivers, or other authorized personnel, while inner siderail control panels 44c are intended to be used by the patient associated with patient support apparatus 20. Each of the control panels 44 includes a plurality of controls 50 (see, e.g. FIGS. 2-3), although each control panel 44 does not necessarily include the same controls and/or functionality.

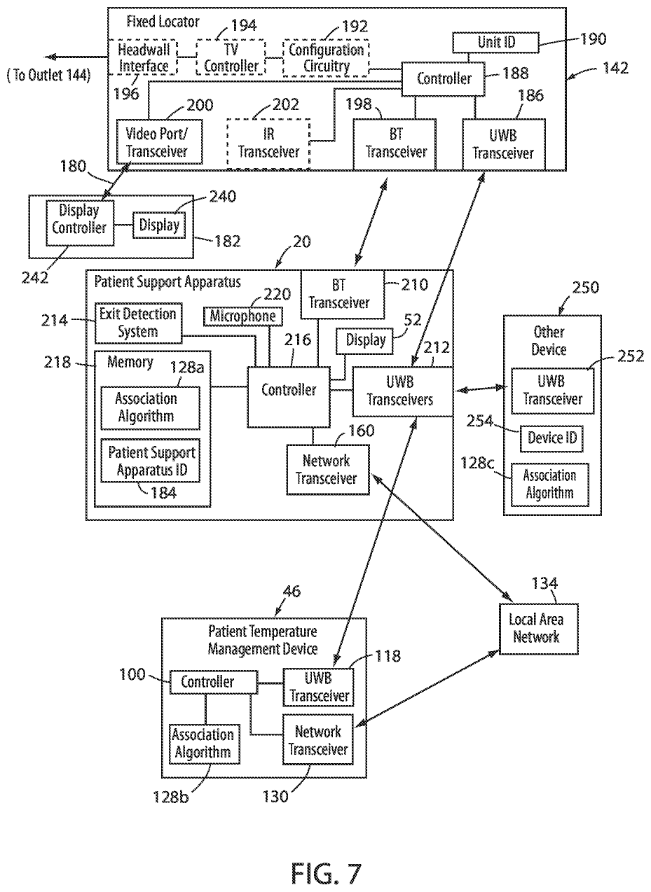

Among other functions, controls 50 of control panel 44a allow a user to control one or more of the following: change a height of support deck 30, raise or lower the Fowler section, activate and deactivate a brake for wheels 24, arm and disarm an exit detection system 214 (FIG. 7), change various settings on patient support apparatus 20, view the current location of the patient support apparatus 20 as determined by the location detection system discussed herein, view data from a patient temperature management device 46 (FIGS. 1 & 4-7) used with the patient, view what medical devices—if any—the patient support apparatus 20 has associated itself with, control what information from the patient temperature management device 46 is displayed and/or sent to an Electronic Medical Records (EMR) server, and perform other actions. One or both of the inner siderail control panels 44c also include at least one control that enables a patient to call a remotely located nurse (or other caregiver). In addition to the nurse call control, one or both of the inner siderail control panels 44c also include one or more controls for controlling one or more features of one or more room devices positioned within the same room as the patient support apparatus 20. As will be described in more detail below, such room devices include, but are not necessarily limited to, a television, a reading light, and a room light. With respect to the television, the features that may be controllable by one or more controls 50 on control panel 44c include, but are not limited to, the volume, the channel, the closed-captioning, and/or the power state of the television. With respect to the room and/or night lights, the features that may be controlled by one or more controls 50 on control panel 44c include the on/off state and/or the brightness level of these lights.

Control panel 44a includes a display 52 (FIG. 2) configured to display a plurality of different screens thereon. Surrounding display 52 are a plurality of navigation controls 50a-f that, when activated, cause the display 52 to display different screens on display 52. More specifically, when a user presses navigation control 50a, control panel 44a displays an exit detection control screen on display 52 that includes one or more icons that, when touched, control the onboard exit detection system 214 (FIG. 7). The exit detection system 214 is as adapted to issue an alert when a patient exits from patient support apparatus 20. Exit detection system 214 may include any of the same features and functions as, and/or may be constructed in any of the same manners as, the exit detection system disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. Other types of exit detection systems may be included within patient support apparatus 20.

When a user presses navigation control 50b (FIG. 2), control panel 44 displays a monitoring control screen that includes a plurality of control icons that, when touched, control an onboard monitoring system built into patient support apparatus 20. The onboard monitoring system alerts the caregiver through a unified indicator, such as a light or a plurality of lights controlled in a unified manner, when any one or more of a plurality of settings on patient support apparatus 20 are in an undesired state, and uses that same unified indicator to indicate when all of the plurality of settings are in their respective desired states. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20.

When a user presses navigation control 50c, control panel 44a displays a scale control screen that includes a plurality of control icons that, when touched, control the scale system of patient support apparatus 20. Such a scale system may include any of the same features and functions as, and/or may be constructed in any of the same manners as, the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system may utilize the same force sensors that are utilized by the exit detection system 214, or it may utilize one or more different sensors. Other scale systems besides those mentioned above in the '254 and '954 applications may alternatively be included within patient support apparatus 20.

When a user presses navigation control 50d, control panel 44 displays a motion control screen that includes a plurality of control icons that, when touched, control the movement of various components of patient support apparatus 20, such as, but not limited to, the height of litter frame 28 and the pivoting of the Fowler section. In some embodiments, the motion control screen displayed on display 52 in response to pressing control 50d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 50e, control panel 44a displays a motion lock control screen that includes a plurality of control icons that, when touched, control one or more motion lockout functions of patient support apparatus 20. Such motion lockout functions typically include the ability for a caregiver to use control panel 44a to lock out one or more of the motion controls 50 of the patient control panels 44c such that the patient is not able to use those controls 50 on control panels 44c to control the movement of one or more components of patient support apparatus 20. The motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

When a user presses on navigation control 50f, control panel 44a displays a menu screen that includes a plurality of menu icons that, when touched, bring up one or more additional screens for controlling and/or viewing one or more other aspects of patient support apparatus 20. Such other aspects include, but are not limited to, displaying information about one or more medical devices that are currently associated with patient support apparatus 20 (e.g. a patient temperature management device, one or more vital sign sensors, etc.), diagnostic and/or service information for patient support apparatus 20, mattress control and/or status information, configuration settings, location information, and other settings and/or information. One example of a suitable menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20.

In at least one embodiment, utilization of navigation control 50f allows a user to navigate to a screen that enables a user to see which medical devices, if any, are currently associated with patient support apparatus 20. In some embodiments, as will be discussed in greater detail herein, patient support apparatus 20 is configured to automatically associate one or more devices with itself when those devices meet certain association conditions, such as being located within a predefined volume of space that encompasses and surrounds patient support apparatus 20 (e.g. volume of space A or B in FIG. 6). As will be discussed in greater detail below, patient support apparatus 20 includes an onboard locating system that is adapted to automatically determine the relative position of one or more devices with respect to patient support apparatus 20 and, in some instances, automatically associate those devices with patient support apparatus 20 (and/or the patient assigned to patient support apparatus 20) depending upon the proximity of the medical device to patient support apparatus 20 and/or other criteria Further details of this locating system and association process are provided below.

For all of the navigation controls 50a-f (FIG. 2), screens other than the ones specifically mentioned above may be displayed on display 52 in other embodiments of patient support apparatus 20 in response to a user pressing these controls. Thus, it will be understood that the specific screens mentioned above are merely representative of the types of screens that are displayable on display 52 in response to a user pressing on one or more of navigation controls 50a-f. It will also be understood that, although navigation controls 50a-f have all been illustrated in the accompanying drawings as dedicated controls that are positioned adjacent display 52, any one or more of these controls 50a-f could alternatively be touchscreen controls that are displayed at one or more locations on display 52. Still further, although controls 50a-f have been shown herein as buttons, it will be understood that any of controls 50a-f could also, or alternatively, be switches, dials, or other types of non-button controls. Additionally, patient support apparatus 20 may be modified to include additional, fewer, and/or different navigation controls from the navigation controls 50a-f shown in FIG. 2.

FIG. 3 illustrates one example of a patient control panel 44c that may be incorporated into patient support apparatus 20 and positioned at a location on patient support apparatus 20 that is convenient for a patient to access while supported on support deck 30, such as on an interior side of one of the siderails 36. Control panel 44c includes a plurality of controls 50g-t that are intended to be operated by a patient. A nurse call control 50g, when pressed by the patient, sends a signal to a nurse call system requesting that a remotely positioned nurse talk to the patient. A Fowler-up control 50h, when pressed by the patient, causes a motorized actuator onboard patient support apparatus 20 to raise the Fowler section upwardly. A Fowler-down control 50i, when pressed by the patient, causes the motorized actuator to lower the Fowler section downwardly. A gatch-up control 50j, when pressed by the patient, causes another motorized actuator to raise a knee section of support deck 30, while a gatch-down control 50k causes the motorized actuator to lower the knee section of support deck 30.

A volume-up control 50l, when pressed by the patient, causes patient support apparatus 20 to send a signal to an in-room television instructing it to increase its volume, while a volume down control 50m, when pressed, causes patient support apparatus 20 to send a signal to the television instructing it to decrease its volume. A channel-up control 50n, when pressed by the patient, causes patient support apparatus 20 to send a signal to the television instructing it to increase the channel number, while a channel-down control 500, when pressed, causes patient support apparatus 20 to send a signal to the television instructing it to decrease the channel number.

A mute control 50p, when pressed, causes patient support apparatus 20 to send a signal to the television instructing it to either mute itself or unmute itself, depending upon whether the television is currently muted or unmuted. In other words, mute control 50p is a toggle control that alternatingly sends mute and unmute commands to the television when it is pressed.

Power control 50q is a toggle control that, when pressed, sends a signal to the television to either turn on or turn off, depending upon the television's current power status. Closed-captioning control 50r is another toggle control that, when pressed, sends a signal to the television to either turn on its closed-captioning feature or to turn off its closed captioning feature, depending upon whether the closed-captioning feature is currently on or off.

Control 50s is a toggle control that, when pressed, sends a signal to a first light to either turn on or turn off, depending upon the current state of that first light. Control 50t is another toggle control that, when pressed, sends a signal to a second light to either turn on or turn off, depending upon the current state of that second light. In some embodiments, the first light is a reading light and the second light is a room light, both of which are positioned off-board the patient support apparatus 20.

It will be understood that not only the number of controls 50 on control panel 44c, but also the functions of the controls 50 on control panel 44c, the layout of the controls 50 on control panel 44c, and/or other aspects of control panel 44c may be modified from what is shown in FIG. 3. In some embodiments, control panel 44c is implemented on a pendant controller that includes a cable that is plugged into a port on patient support apparatus 20. In other embodiments, one or more of the controls 50 of control panel 44c may be omitted, augmented, and/or split amongst other controls panels and/or locations. Still other manners of implementing control panel 44c are also possible.

Thermal control system 48 (FIG. 1) is adapted to control the temperature of a patient, which may involve raising, lowering, and/or maintaining the patient's temperature. Thermal control system 48 includes a patient temperature management device 46 coupled to one or more thermal therapy devices 54. The thermal therapy devices 54 are illustrated in FIG. 1 to be thermal wraps, but it will be understood that thermal therapy devices 54 may take on other forms, such as, but not limited to, blankets, vests, patches, caps, catheters, or other structures that receive temperature-controlled fluid. For purposes of the following written description, thermal therapy devices 54 will be referred to as thermal wraps 54, but it will be understood by those skilled in the art that this terminology is used merely for convenience and that the phrase “thermal wrap” is intended to cover all of the different variations of thermal therapy devices 54 mentioned above (e.g. blankets, vests, patches, pads, caps, catheters, etc.) and variations thereof.

Patient temperature management device 46 is coupled to thermal wraps 54 via a plurality of hoses 56. Patient temperature management device 46 delivers temperature-controlled fluid (such as, but not limited to, water or a water mixture) to the thermal wraps 54 via the fluid supply hoses 56. After the temperature-controlled fluid has passed through thermal wraps 54, patient temperature management device 46 receives the temperature-controlled fluid back from thermal wraps 54 via the return hoses 56b.

In the embodiment of thermal control system 48 shown in FIG. 1, three thermal wraps 54 are used in the treatment of the patient. A first thermal wrap 54 is wrapped around a patient's torso, while second and third thermal wraps 54 are wrapped, respectively, around the patient's right and left legs. Other configurations can be used and different numbers of thermal wraps 54 may be used with patient temperature management device 46, depending upon the number of inlet and outlet ports that are included with patient temperature management device 46. By controlling the temperature of the fluid delivered to thermal wraps 54 via supply hoses 56a, the temperature of the patient can be controlled via the close contact of the wraps 54 with the patient and the resultant heat transfer therebetween.

As shown more clearly in FIG. 4, patient temperature management device 46 includes a main body 58 to which a removable reservoir 60 may be coupled and uncoupled. Removable reservoir 60 is configured to hold the fluid that is to be circulated through patient temperature management device 46 and the one or more thermal wraps 54. By being removable from patient temperature management device 46, reservoir 60 can be easily carried to a sink or faucet for filling and/or dumping of the water or other fluid. This allows users of thermal control system 48 to more easily fill patient temperature management device 46 prior to its use, as well as to drain patient temperature management device 46 after use.

As can also be seen in FIG. 4, patient temperature management device 46 includes a plurality of outlet ports 62 (three in the particular example of FIG. 4), a plurality of inlets ports 64 (three in this particular example). Outlet ports 62 are adapted to fluidly couple to supply hoses 56a and inlet ports 64 are adapted to fluidly couple to return hoses 56b. Patient temperature management device 46 also includes a plurality of patient temperature probe ports 66, a plurality of auxiliary ports 68, and a control panel 70 having a plurality of dedicated controls 72 and a display 74 (see also FIGS. 1 & 5). The patient temperature probe ports 66, auxiliary ports 68, and control panel 70 are described in more detail below.

As shown in FIG. 5, patient temperature management device 46 includes a pump 76 for circulating fluid through a circulation channel 78. Pump 76, when activated, circulates the fluid through circulation channel 78 in the direction of arrows 80 (clockwise in FIG. 5). Starting at pump 76 the circulating fluid first passes through a heat exchanger 82 that adjusts, as necessary, the temperature of the circulating fluid. Heat exchanger 82 may take on a variety of different forms. In some embodiments, heat exchanger 82 is a thermoelectric heater and cooler. In the embodiment shown in FIG. 5, heat exchanger 82 includes a chiller 84 and a heater 86. Further, in the embodiment shown in FIG. 5, chiller 84 is a conventional vapor-compression refrigeration unit having a compressor 88, a condenser 90, an evaporator 92, an expansion valve (not shown), and a fan 94 for removing heat from the compressor 88. Heater 86 is a conventional electrical resistance-based heater. Other types of chillers and/or heaters may be used.

After passing through heat exchanger 82, the circulating fluid is delivered to an outlet manifold 96 having an outlet temperature sensor 98 and a plurality of outlet ports 62. Temperature sensor 98 is adapted to detect a temperature of the fluid inside of outlet manifold 96 and report it to a controller 100. Outlet ports 62 are adapted to releasably couple to supply hoses 56a. Supply hoses 56a are adapted to releasably couple, in turn, to thermal wraps 54 and deliver temperature-controlled fluid to the thermal wraps 54. The temperature-controlled fluid, after passing through the thermal wraps 54, is returned to patient temperature management device 46 via return hoses 56b. Return hoses 56b are adapted to be releasably coupled to a plurality of inlets ports 64. The releasable coupling between hoses 56, ports 62 and 64 of patient temperature management device 46, and thermal wraps 54 may take place via any suitable means, such as a conventional Colder-type connector, or another type of connector. Inlets ports 64 are fluidly coupled to an inlet manifold 102 inside of patient temperature management device 46.

Patient temperature management device 46 also includes a bypass line 104 fluidly coupled to outlet manifold 96 and inlet manifold 102 (FIG. 5). Bypass line 104 allows fluid to circulate through circulation channel 78 even in the absence of any thermal wraps 54 or hoses 56a being coupled to any of outlet ports 62. In the illustrated embodiment, bypass line 104 includes a filter 106 that is adapted to filter the circulating fluid. If included, filter 106 may be a particle filter adapted to filter out particles within the circulating fluid that exceed a size threshold, or filter 106 may be a biological filter adapted to purify or sanitize the circulating fluid, or it may be a combination of both. In some embodiments, filter 106 is constructed and/or positioned within patient temperature management device 46 in any of the manners disclosed in commonly assigned U.S. patent application Ser. No. 62/404,676 filed Oct. 11, 2016, by inventors Marko Kostic et al. and entitled THERMAL CONTROL SYSTEM, the complete disclosure of which is incorporated herein by reference.

The flow of fluid through bypass line 104 is controllable by way of a bypass valve 108 positioned at the intersection of bypass line 104 and outlet manifold 96 (FIG. 5). When open, bypass valve 108 allows fluid to flow through circulation channel 78 to outlet manifold 96, and from outlet manifold 96 to the connected thermal wraps 54. When closed, bypass valve 108 stops fluid from flowing to outlet manifold 96 (and thermal wraps 54) and instead diverts the fluid flow along bypass line 104. In some embodiments, bypass valve 108 may be controllable by controller 100 such that selective portions of the fluid are directed to outlet manifold 96 and along bypass line 104. In some embodiments, bypass valve 108 is controlled in any of the manners discussed in commonly assigned U.S. patent application Ser. No. 62/610,319, filed Dec. 26, 2017, by inventors Gregory Taylor et al. and entitled THERMAL SYSTEM WITH OVERSHOOT REDUCTION, the complete disclosure of which is incorporated herein by reference. In other embodiments, bypass valve 108 may be a pressure operated valve that allows fluid to flow along bypass line 104 if the fluid pressure in circulation channel 78 exceeds the cracking pressure of the bypass valve 108. Still further, in some embodiments, bypass valve 108 may be omitted and fluid may be allowed to flow through both bypass line 104 and into outlet manifold 96.

The incoming fluid flowing into inlet manifold 102 from inlets ports 64 and/or bypass line 104 travels back toward pump 76 and into an air remover 110. Air remover 110 includes any structure in which the flow of fluid slows down sufficiently to allow air bubbles contained within the circulating fluid to float upwardly and escape to the ambient surroundings. In some embodiments, air remover 110 is constructed in accordance with any of the configurations disclosed in commonly assigned U.S. patent application Ser. No. 15/646,847 filed Jul. 11, 2017, by inventor Gregory S. Taylor and entitled THERMAL CONTROL SYSTEM, the complete disclosure of which is hereby incorporated herein by reference. After passing through air remover 110, the circulating fluid flows past a valve 112 positioned beneath fluid reservoir 60. Fluid reservoir 60 supplies fluid to patient temperature management device 46 and circulation channel 78 via valve 112, which may be a conventional check valve, or other type of valve, that automatically opens when reservoir 60 is coupled to patient temperature management device 46 and that automatically closes when reservoir 60 is decoupled from patient temperature management device 46 (see FIG. 4). After passing by valve 112, the circulating fluid travels to pump 76 and the fluid circuit is repeated.

Controller 100 of patient temperature management device 46 is contained within main body 58 of patient temperature management device 46 and is in electrical communication with pump 76, heat exchanger 82, outlet temperature sensor 98, bypass valve 108, a sensor module 114, control panel 70, a memory 116, a network transceiver 130, and one or more ultra-wideband transceivers 118. Controller 100 includes any and all electrical circuitry and components necessary to carry out the functions and algorithms described herein, as would be known to one of ordinary skill in the art. Generally speaking, controller 100 may include one or more microcontrollers, microprocessors, and/or other programmable electronics that are programmed to carry out the functions described herein. It will be understood that controller 100 may also include other electronic components that are programmed to carry out the functions described herein, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions in patient temperature management device 46, or they may reside in a common location within patient temperature management device 46. When physically distributed, the components may communicate using any suitable serial or parallel communication protocol, such as, but not limited to, CAN, LIN, Firewire, I-squared-C, RS-232, RS-465, universal serial bus (USB), etc.

Control panel 70 allows a user to operate patient temperature management device 46. Control panel 70 communicates with controller 100 and includes a display 74 and a plurality of dedicated controls 72a, 72b, 72c, etc. Display 74 may be implemented as a touch screen, or, in other embodiments, as a non-touch-sensitive display. Dedicated controls 72 may be implemented as buttons, switches, dials, or other dedicated structures. In any of the embodiments, one or more of the functions carried out by a dedicated control 72 may be replaced or supplemented with a touch screen control that is activated when touched by a user. Alternatively, in any of the embodiments, one or more of the controls that are carried out via a touch screen can be replaced or supplemented with a dedicated control 72 that carries out the same function when activated by a user.

Through either dedicated controls 72 and/or a touch screen display (e.g. display 74), control panel 70 enables a user to turn patient temperature management device 46 on and off, select a mode of operation, select a target temperature for the fluid delivered to thermal wraps 54, select a patient target temperature, customize a variety of treatment, display, alarm, and other functions, view the association status of patient temperature management device 46 with patient support apparatus 20 and/or with hoses 56 and/or thermal wraps 54, control what information from patient temperature management device 46 is sent to patient support apparatus 20, and control still other aspects of patient temperature management device 46, as is discussed in greater detail below. In some embodiments, control panel 70 may include a pause/event control, a medication control, and/or an automatic temperature adjustment control that operate in accordance with the pause event control 66b, medication control 66c, and automatic temperature adjustment control 66d disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. Such controls may be activated as touch screen controls or dedicated controls 72.

In those embodiments where control panel 70 allows a user to select from different modes for controlling the patient's temperature, the different modes include, but are not limited to, a manual mode and an automatic mode, both of which may be used for cooling and heating the patient. In the manual mode, a user selects a target temperature for the fluid that circulates within patient temperature management device 46 and that is delivered to thermal wraps 54. Patient temperature management device 46 then makes adjustments to heat exchanger 82 in order to ensure that the temperature of the fluid exiting supply hoses 56a is at the user-selected temperature.

When the user selects the automatic mode, the user selects a target patient temperature, rather than a target fluid temperature. After selecting the target patient temperature, controller 100 makes automatic adjustments to the temperature of the fluid in order to bring the patient's temperature to the desired patient target temperature. In this mode, the temperature of the circulating fluid may vary as necessary in order to bring about the target patient temperature.

In order to carry out the automatic mode, patient temperature management device 46 utilizes a sensor module 114 that includes one or more patient temperature sensor ports 66 (FIGS. 4 & 5) that are adapted to receive one or more conventional patient temperature sensors or probes 87. The patient temperature sensors 87 may be any suitable patient temperature sensor that is able to sense the temperature of the patient at the location of the sensor. In one embodiment, the patient temperature sensors are conventional Y.S.I. 400 probes marketed by YSI Incorporated of Yellow Springs, Ohio, or probes that are YSI 400 compliant or otherwise marketed as 400 series probes. In other embodiments, different types of sensors may be used with patient temperature management device 46. Regardless of the specific type of patient temperature sensor used in thermal control system 48, each temperature sensor 87 is connected to a patient temperature sensor port 66 positioned on patient temperature management device 46. Patient temperature sensor ports 66 are in electrical communication with controller 100 and provide current temperature readings of the patient's temperature.

Controller 100, in some embodiments, controls the temperature of the circulating fluid using closed-loop feedback from temperature sensor 98 (and, when operating in the automatic mode, also from patient temperature sensor(s) 87). That is, controller 100 determines (or receives) a target temperature of the fluid, compares it to the measured temperature from sensor 98, and issues a command to heat exchanger 82 that seeks to decrease the difference between the desired fluid temperature and the measured fluid temperature. In some embodiments, the difference between the fluid target temperature and the measured fluid temperature is used as an error value that is input into a conventional Proportional, Integral, Derivative (PID) control loop. That is, controller 100 multiplies the fluid temperature error by a proportional constant, determines the derivative of the fluid temperature error over time and multiplies it by a derivative constant, and determines the integral of the fluid temperature error over time and multiplies it by an integral constant. The results of each product are summed together and converted to a heating/cooling command that is fed to heat exchanger 82 and tells heat exchanger 82 whether to heat and/or cool the circulating fluid and how much heating/cooling power to use.

When patient temperature management device 46 is operating in the automatic mode, controller 100 may use a second closed-loop control loop that determines the difference between a patient target temperature and a measured patient temperature. The patient target temperature is input by a user of patient temperature management device 46 using control panel 70. The measured patient temperature comes from a patient temperature sensor 87 coupled to one of patient temperature sensor ports 66 (FIG. 5). Controller 100 determines the difference between the patient target temperature and the measured patient temperature and, in some embodiments, uses the resulting patient temperature error value as an input into a conventional PID control loop. As part of the PID loop, controller 100 multiplies the patient temperature error by a proportional constant, multiplies a derivative of the patient temperature error over time by a derivative constant, and multiplies an integral of the patient temperature error over time by an integral constant. The three products are summed together and converted to a target fluid temperature value. The target fluid temperature value is then fed to the first control loop discussed above, which uses it to compute a fluid temperature error.

It will be understood by those skilled in the art that other types of control loops may be used. For example, controller 100 may utilize one or more PI loops, PD loops, and/or other types of control equations. In some embodiments, the coefficients used with the control loops may be varied by controller 100 depending upon the patient's temperature reaction to the thermal therapy, among other factors. One example of such dynamic control loop coefficients is disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference.

Regardless of the specific control loop utilized, controller 100 implements the loop(s) multiple times a second in at least one embodiment, although it will be understood that this rate may be varied widely. After controller 100 has output a heat/cool command to heat exchanger 82, controller 100 takes another patient temperature reading (from sensor 87) and/or another fluid temperature reading (from sensor 98) and re-performs the loop(s). The specific loop(s) used, as noted previously, depends upon whether patient temperature management device 46 is operating in the manual mode or automatic mode.

It will also be understood by those skilled in the art that the output of any control loop used by patient temperature management device 46 may be limited such that the temperature of the fluid delivered to thermal wraps 54 never strays outside of a predefined maximum and a predefined minimum. Examples of such a predefined maximum temperature and predefined minimum temperature are disclosed and discussed in greater detail in commonly assigned U.S. patent application Ser. No. 16/222,004 filed Dec. 17, 2018, by inventors Gregory S. Taylor et al. and entitled THERMAL SYSTEM WITH GRAPHICAL USER INTERFACE, the complete disclosure of which is incorporated herein by reference. The predefined minimum temperature is designed as a safety temperature and may be set to about four degrees Celsius, although other temperatures may be selected. The predefined maximum temperature is also implemented as a safety measure and may be set to about forty degrees Celsius, although other values may be selected.

In some embodiments of patient temperature management device 46, such as the embodiment shown in FIG. 5, patient temperature management device 46 also includes a reservoir valve 120 that is adapted to selectively move fluid reservoir 60 into and out of line with circulation channel 78. Reservoir valve 120 is positioned in circulation channel 78 between air remover 110 and valve 112, although it will be understood that reservoir valve 120 may be moved to different locations within circulation channel 78. Reservoir valve 120 is coupled to circulation channel 78 as well as a reservoir channel 122. When reservoir valve 120 is open, fluid from air remover 110 flows along circulation channel 78 to pump 76 without passing through reservoir 60 and without any fluid flowing along reservoir channel 122. When reservoir valve 120 is closed, fluid coming from air remover 110 flows along reservoir channel 122, which feeds the fluid into reservoir 60. Fluid inside of reservoir 60 then flows back into circulation channel 78 via valve 112. Once back in circulation channel 78, the fluid flows to pump 76 and is pumped to the rest of circulation channel 78 and thermal wraps 54 and/or bypass line 104. In some embodiments, reservoir valve 120 is either fully open or fully closed, while in other embodiments, reservoir valve 120 may be partially open or partially closed. In either case, reservoir valve 120 is under the control of controller 100.

In those embodiments of patient temperature management device 46 that include a reservoir valve 120, patient temperature management device 46 may also include a reservoir temperature sensor 124. Reservoir temperature sensor 124 reports its temperature readings to controller 100. When reservoir valve 120 is open, the fluid inside of reservoir 60 stays inside of reservoir 60 (after the initial drainage of the amount of fluid needed to fill circulation channel 78 and thermal wraps 54). This residual fluid is substantially not affected by the temperature changes made to the fluid within circulation channel 78 as long as reservoir valve 120 remains open. This is because the residual fluid that remains inside of reservoir 60 after circulation channel 78 and thermal wraps 54 have been filled does not pass through heat exchanger 82 and remains substantially thermally isolated from the circulating fluid. Two results flow from this: first, heat exchanger 82 does not need to expend energy on changing the temperature of the residual fluid in reservoir 60, and second, the temperature of the circulating fluid in circulation channel 78 will deviate from the temperature of the residual fluid as the circulating fluid circulates through heat exchanger 82.

In some embodiments, controller 100 utilizes a temperature control algorithm to control reservoir valve 120 that, in some embodiments, is the same as the temperature control algorithm 160 disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. In other embodiments, controller 100 utilizes a different control algorithm. In still other embodiments, patient temperature management device 46 is modified to omit reservoir valve 120, reservoir channel 122, and reservoir temperature sensor 124. Patient temperature management device 46 may also be modified such that reservoir 60 is always in the path of circulation channel 78. Still other modifications are possible.

It will be understood that the particular order of the components along circulation channel 78 of patient temperature management device 46 may be varied from what is shown in FIG. 5. For example, although FIG. 5 depicts pump 76 as being upstream of heat exchanger 82 and air separator 110 as being upstream of pump 76, this order may be changed. Air separator 110, pump 76, heat exchanger 82 and reservoir 60 may be positioned at any suitable location along circulation channel 78. Indeed, in some embodiments, reservoir 60 is moved so as to be in line with and part of circulation channel 78, rather than external to circulation channel 78 as shown in FIG. 5, thereby forcing the circulating fluid to flow through reservoir 60 rather than around reservoir 60. It will also be understood that patient temperature management device 46 does not need to include all of the components shown in FIG. 5, and that many embodiments of patient temperature management device 46 may be implemented in accordance with the present disclosure that omit one or more of these illustrated components. Further details regarding the construction and operation of one embodiment of patient temperature management device 46 that are not described herein may be found in commonly assigned U.S. patent application Ser. No. 14/282,383 filed May 20, 2014, by inventors Christopher Hopper et al. and entitled THERMAL CONTROL SYSTEM, the complete disclosure of which is incorporated herein by reference.

In some embodiments, thermal wraps 54 (FIGS. 1, 5-6) are constructed in accordance with any of the thermal pads disclosed in any of the following commonly assigned U.S. patent applications: Ser. No. 15/675,061 filed Aug. 11, 2017, by inventors James Galer et al. and entitled THERMAL THERAPY DEVICES; Ser. No. 62/778,034 filed Dec. 11, 2018, by inventors Andrew M. Bentz et al. and entitled THERMAL SYSTEM WITH THERMAL PAD FILTERS; and Ser. No. 15/675,066 filed Aug. 11, 2017, by inventor James K. Galer and entitled THERMAL SYSTEM, the complete disclosures of all of which are incorporated herein by reference. Still other types of thermal wraps 54 may be used with thermal control system 48, and patient temperature management device 46 may be modified from its construction described herein in order to accommodate the particular thermal therapy pad(s) it is used with.

Memory 116 (FIG. 5) may be any type of conventional non-volatile memory, such as, but not limited to flash memory, one or more hard drives, one or more EEPROMs, etc. Memory 116 may also be implemented to include more than one of these types of memories in combination. In the embodiment shown in FIG. 5, memory 116 of patient temperature management device 46 includes, among other information, a unique ID 126 and an association algorithm 128a. Unique ID 126 uniquely identifies patient temperature management device 46 and distinguishes it from any other patient temperature management devices 46 (and other devices) that may be present within a given healthcare facility. Association algorithm 128a is used by controller 100, in some embodiments, to determine whether to associate temperature management device 46 with a patient support apparatus 20 or with a fixed locator 142 (FIG. 6), and/or to make other association and/or data-sharing decisions, as will be discussed in greater detail below. Memory 116 may also include additional information beyond that shown in FIG. 5, such as, but not limited to, one or more additional algorithms for carrying out its functions, data recorded during the operation of patient temperature management device 46, and/or other data.

Network transceiver 130 is adapted to communicate with one or more off-board devices, such as, but not limited to, a wireless access point of a local area network, a network cable of a local area network, and/or other devices. In the embodiment shown in FIG. 5, transceiver 130 is a Wi-Fi radio communication module configured to wirelessly communicate with one or more wireless access points 132 of a local area network 134 (FIGS. 5-6). In such embodiments, transceiver 130 may operate in accordance with any of the various IEEE 802.11 standards (e.g. 802.11b, 802.11n, 802.11 g, 802.11ac, 802.11ah, etc.). In other embodiments, transceiver 130 may include, either additionally or in lieu of the Wi-Fi radio and communication module, a wired port for connecting a network wire to patient temperature management device 46. In some such embodiments, the wired port accepts a category 5e cable (Cat-5e), a category 6 or 6a (Cat-6 or Cat-6a), a category 7 (Cat-7) cable, or some similar network cable, and transceiver 130 is an Ethernet transceiver. In still other embodiments, transceiver 130 may be constructed to include the functionality of the communication modules 56 disclosed in commonly assigned U.S. patent application Ser. No. 15/831,466 filed Dec. 5, 2017, by inventor Michael Hayes et al. and entitled NETWORK COMMUNICATION FOR PATIENT SUPPORT APPARATUSES, the complete disclosure of which is incorporated herein by reference.

Regardless of the specific structure included with transceiver 130, controller 100 is able to communicate with the local area network 134 (FIGS. 5-6) of a healthcare facility in which the patient temperature management device 46 is positioned. When transceiver 130 is a wireless transceiver, it communicates with local area network 134 via one or more wireless access points 132. When transceiver 130 is a wired transceiver, it communicates directly via a cable coupled between patient temperature management device 46 and a network outlet positioned within the room of the healthcare facility in which patient temperature management device 46 is positioned.

Local area network 134 typically includes a plurality of servers, the contents of which will vary from healthcare facility to healthcare facility. In general, however, most healthcare facilities will include, among other servers, an electronic medical records (EMR) server 136, which may be a conventional server. In addition to EMR server 136, local area network 134 may include a patient support apparatus server 138 that is in communication with one or more patient support apparatuses 20 and/or patient temperature management devices 46 positioned within the healthcare facility. Server 138 may also be communicatively coupled (via the Internet or other means) to one or more other servers that are positioned outside of the healthcare facility. Further details regarding network 134 and the servers that may be hosted thereon are discussed below with respect to FIG. 6.

Although not shown in FIG. 5, patient temperature management device 46 may include a clock/calendar (not shown) that communicates with controller 100. The clock/calendar not only measures the passage of time, but it also keeps track of the calendar day (and year). The clock/calendar may be any conventional timing device that is able to keep track of the passage of time, including the calendar day and year.

In addition to the patient temperature sensor(s) 87, the water temperature sensor 98, and the reservoir temperature sensor 124 (if included), patient temperature management device 46 may include still more sensors that are positioned within main body 58, and/or that are positioned outside of main body 58 and in communication with main controller 100. Such off-board sensors (e.g. outside of main body 58) may communicate with main controller 100 via one or more of the auxiliary sensor ports 68 and/or via one or more of the transceivers 130. Each auxiliary sensor port 68 is adapted to receive outputs from an off-board auxiliary sensor 140. The auxiliary sensors 140, as well as any additional sensors onboard patient temperature management device 46, provide additional data to controller 100 regarding the patient during a thermal therapy session. Controller 100 is configured to utilize the additional data either for use in one or more algorithms that are currently being used by patient temperature management device 46 to control the patient's temperature, or for potential future use in one or more improved algorithms that are determined, after analysis, to provide improved results for the thermal therapy sessions carried out using patient temperature management device 46.

Auxiliary ports 68 (FIGS. 4 & 5) may take on a variety of different forms. In one embodiment, all of the ports 68 (if there are more than one) are of the same type. In another embodiment, patient temperature management device 46 includes multiple types of ports. In any of these embodiments, the ports 68 may include, but are not limited to, a Universal Serial Bus (USB) port, an Ethernet port (e.g. an 8P8C modular connector port, or the like), a parallel port, a different (from USB) type of serial port, etc. Ports 68 may also or alternatively be implemented wirelessly, such as via a WiFi transceiver, a Bluetooth transceiver, a ZigBee transceiver, etc.

Patient temperature management device 46 may be configured to accept a number of different types of auxiliary sensors 140 via input ports 68. Such sensors include, but are not limited to, the sensors 128 disclosed in commonly assigned PCT patent application serial number PCT/US2021/061947 filed Dec. 6, 2020, by applicant Stryker Corporation and entitled THERMAL CONTROL SYSTEMS WITH DYNAMIC CONTROL ALGORITHMS, the complete disclosure of which is incorporated herein by reference. In some embodiments, patient temperature management device 46 omits auxiliary ports 68 and is not configured to accept any auxiliary sensors 140.

FIG. 6 illustrates patient support apparatus 20 and thermal control system 48 positioned within a room of a healthcare facility. FIG. 6 also illustrates several additional items that may be present in a healthcare facility and which patient support apparatus 20 and/or patient temperature management device 46 are configured to communicate with, including, but not limited to, a locator unit 142 and a conventional local area network 134 of the healthcare facility. Locator units 142 are positioned at known and fixed locations within the healthcare facility in which patient support apparatus 20 are typically positioned. Locator units 142 function as fixed locators. That is, locator units 142 communicate with patient support apparatuses 20, patient temperature management devices 46, and/or other devices and share information with them that allows the location of those devices to be determined.

In some embodiments, two different types of locator units 142 may be present within a healthcare facility: linked locator units and unlinked locator units. The linked and unlinked locator units 142 differ from each other in that the linked locator units are adapted to communicate with a conventional communication outlet 144 that is typically built into one or more walls of a healthcare facility. That is, the linked locator units 142 are communicatively linked to a conventional communication outlet 144. The unlinked locator units 142 are not adapted to communicate with such communication outlets 144, and are therefore not linked to a nearby communications outlet 144. Both types of locator units are adapted to provide location information to patient support apparatus 20, patient temperature management device 46, and/or other types of devices. The linked locator units 142, however, are also adapted to serve as a communication conduit for routing communications between any devices and/or systems that are desired to be in communication with communication outlet 144 (e.g. a reading light 146, a room light 148, a television 150, and/or a nurse call system 152 (FIG. 6)). The unlinked locator units 142, in contrast, are not adapted to serve as communication conduits between a communication outlet 144 and these types of devices. In general, the linked locator units 142 are typically positioned in patient rooms of the healthcare facility where one or more communication outlets 144 are typically present, while the unlinked locator units 142 are typically positioned in locations outside of patient rooms, such as hallways, maintenance areas, and/or other areas. Unless explicitly stated otherwise, references herein to “locator units 142” refer to both types of locator units.

As shown in FIG. 6, locator units 142 are adapted to be mounted to a wall 154, such as a headwall of a patient room within the healthcare facility. The headwall of a conventional healthcare facility room typically includes a conventional communications outlet 144 physically integrated therein. Communications outlet 144 is adapted to receive a conventional nurse call cable 156 that physically connects at its other end either to patient support apparatus 20 (not shown) or to locator unit 142 (shown in FIG. 6). In many healthcare facilities, communication outlet 144 includes a 37-pin connector, although other types of connectors are often found in certain healthcare facilities. As will be discussed in greater detail below, linked locator unit 142 and nurse call cable 156 allow patient support apparatus 20 to communicate with a nurse call system, and one or more room devices positioned within room.

Communication outlet 144 is electrically coupled to one or more cables, wires, or other conductors 158 that electrically couple the communication outlet 144 to a nurse call system 152 and one or more conventional room devices, such as a reading light 146, a room light 148, and/or a television 150. Conductors 158 are typically located behind the wall of the room and not visible to people in the room. In some healthcare facilities, conductors 158 may first couple to a room interface circuit board that includes one or more conductors 158 for electrically coupling the room interface circuit board to room devices 146, 148, 150 and/or nurse call system 152. Still other communicative arrangements for coupling communication outlet 144 to nurse call system 152 and/or one or more room devices 146, 148, and 150 are possible.

Nurse call cable 156 (FIG. 6) enables locator unit 142 to communicate with nurse call system 152 and/or room devices 146, 148, 150, and because patient support apparatus 20 is able to wirelessly communicate with locator unit 142, patient support apparatus 20 is thereby able to communicate with nurse call system 152 and room devices 146, 148, 150. A patient supported on patient support apparatus 20 who activates a nurse call control (e.g. 50g; see FIG. 3) on patient support apparatus 20 causes a signal to be wirelessly sent from patient support apparatus 20 to locator unit 142, which in turn conveys the signal via nurse call cable 156 to the nurse call system 152, which forwards the signal to one or more remotely located nurses (e.g. nurses at one or more nurse's stations 174). If the patient activates one or more room device controls (e.g. controls 50l-t; see FIG. 3), one or more wireless signals are conveyed to the locator unit 142, which in turn sends appropriate signals via nurse call cable 156 to communication outlet 144 and the room device 146, 148, 150 that change one or more features of these devices (e.g. the volume, channel, on/off state, etc.).

As is also shown in FIG. 6, patient support apparatus 20 is further configured to communicate with a local area network 134 of the healthcare facility. In the embodiment shown in FIG. 6, patient support apparatus 20 includes a wireless network transceiver 160 (FIG. 7) that communicates wirelessly with local area network 134. Network transceiver 160 is, in at least some embodiments, a WiFi transceiver (e.g. IEEE 802.11) that wirelessly communicates with one or more conventional wireless access points 132 of local area network 134. In other embodiments, network transceiver 160 may be a wireless transceiver that uses conventional 5G technology to communicate with network 134, one or more servers hosted thereon, and/or other devices. In some embodiments, network transceiver 160 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In some embodiments, network transceiver 160 is a wired transceiver that is adapted to allow patient support apparatus 20 to communicate with network 134 via a wired connection, such as an Ethernet cable that plugs into an Ethernet port (e.g. an RJ-45 style port, an 8P8C port, etc.) built into patient support apparatus 20. In still other embodiments, patient support apparatus 20 includes both a wired transceiver 160 for communicating with network 134 via a wired connection and a wireless transceiver 160 for wirelessly communicating with network 134.

Patient support apparatus 20 is configured to communicate with one or more servers on local area network 134 of the healthcare facility. One such server is a patient support apparatus server 138. Patient support apparatus server 138 is adapted, in at least one embodiment, to receive status information from patient support apparatuses 20 positioned within the healthcare facility and distribute this status information to caregivers, other servers, and/or other software applications. As will be discussed in greater detail below, server 138 may also be configured to receive data from one or more other devices (such as, but not limited to, patient temperature management device 46) that are associated either with patient support apparatus 20 or a locator unit 142. Additionally, in some embodiments, the data from one or more of devices may be forwarded by server 138 to one or more electronic devices 162 and/or to one or more other servers on network 134.

In some embodiments, patient support apparatus server 138 is configured to communicate at least some of the devices associated with a patient support apparatus 20 and/or a locator unit 142 to a remote server 164 that is positioned geographically remotely from the healthcare facility. Such communication may take place via a conventional network appliance 166, such as, but not limited to, a router and/or a gateway, that is coupled to the Internet 168. The remote server 164, in turn, is also coupled to the Internet 168, and patient support apparatus server 138 is provided with the URL and/or other information necessary to communicate with remote server 164 via the Internet connection between network 134 and server 164.

In some alternative embodiments, patient support apparatus 20, temperature management device 46, and/or other devices of the present disclosure may be configured to communicate directly with one or more cloud-based servers, such as remote server 164, without utilizing patient support apparatus server 138. That is, in some embodiments, patient support apparatuses 20 may be configured to communicate directly with a remote server without relying upon any locally hosted servers (e.g. servers hosted on network 134). In one such embodiment, patient support apparatus 20 utilizes Microsoft's Azure could computing service to directly connect to one or more remote servers 164 without utilizing server 138. In some such embodiments, network appliance 166 is a router configured to support such direct connections. Still other types of direct-to-cloud connections may be utilized with one or more of patient support apparatuses 20.

Patient support apparatus server 138 is also configured to determine the location of each device that is equipped with one or more ultra-wideband transceivers and that is associated with a locator unit (or with another device that is associated with a locator unit 142). These devices include, but are not limited to, patient support apparatus 20, temperature management device 46, and other devices. For purposes of the following written description, these devices will be referred to generically herein as UWB-devices 250. UWB-devices 250 will therefore generically refer to any device that includes one or more UWB-transceivers and that is configured to use those UWB-transceivers to communicate with a locator unit 142 and/or a patient support apparatus 20 in the manners described below. In some instances, as will be discussed more below, UWB devices 250 utilize an association algorithm 128 for communicating with a patient support apparatus 20 and/or a locator unit 142. In some instances, UWB devices 250 may utilize other algorithms for communicating with patient support apparatus 20, locator unit 142, and/or with other devices.

In some embodiments, patient support apparatus server 138 determines the room number and/or bay area of each UWB device 250 that is positioned within a room, as well as the location of any UWB devices 250 that are positioned outside of a room but within a threshold distance of an unlinked locator unit 142 (e.g. devices 250 that may be positioned in a hallway, a maintenance area, or some other area). In general, patient support apparatus server 138 may be configured to determine the position of any UWB device 250 that is positioned within communication range of one or more locator units 142 and/or within communication range of a patient support apparatus 20 that is, in turn, positioned within communication range of a locator unit 142, as will be discussed in greater detail below.

EMR server 136 (FIG. 6) is a conventional server that stores the patients' electronic medical records. Such electronic medical records typically contain medical information about a patient, such as the patient's treatment, medical history, prescriptions, and/or therapies, assessments, etc. Admissions, Discharge, and Transfer (ADT) server 170, which may be a part of EMR server 136 or a separate server, is a conventional server that keeps track of patients' room assignments and, in some versions, the caregivers assigned to the patients. ADT server 170 is used for managing the admission, discharge, and transfer of patients in the healthcare facility. The ADT server 170 stores patient location information, including the identity of patients and the corresponding rooms (and bay numbers in rooms with more than one patient). Patient support apparatus server 138 is adapted to communicate with both EMR server 136 and ADT server 170.

In some embodiments, patient support apparatus server 138 (FIG. 6) is adapted to determine which patient is assigned to a particular room by communicating with ADT server. Additionally, patient support apparatus server 138 is adapted to store, or have access to, a table that lists the locations of each fixed locator unit 142 within the healthcare facility. When a UWB device 250 reports the unique ID of the fixed locator 142 that it is currently associated with to patient support apparatus server 138, patient support apparatus server 138 uses this fixed locator ID to determine in which room or bed bay the UWB device 250 is currently located. From this room number and/or bed bay number, patient support apparatus server 138 is adapted to determine which patient is assigned to a UWB device 250 by requesting from ADT server 170 the identity of the patient who is currently assigned to that particular room and/or bay number. Once patient support apparatus server 138 determines which patient is assigned to a particular room and/or bay, it concludes that any UWB devices 250 that are currently positioned within that room (and/or bay) are to be assigned to that particular patient, and therefore associates those UWB devices with the particular patient assigned to that room and/or bay. This association allows server 138 to know which medical record the data from the UWB devices 250 is to be recorded in. It also allows server 138 to know which caregiver is associated with a particular patient, and to therefore route notifications to appropriate personnel, as will be discussed in greater detail below. Still further it, it allows server 138 to know which display devices and/or electronic devices 162 (if any) all or some of the data is to be displayed on, as will also be discussed in greater detail below.

The routing of data from a UWB device 250 to patient support apparatus server 138 can occur in at least three different manners. In a first manner, the UWB device 250 transmits its data directly to a patient support apparatus 20 with which the UWB device 250 is associated (such association is discussed in greater detail below). Such transmission of data to an associated patient support apparatus 20 may take place via a Bluetooth transceiver, an ultra-wideband transceiver, or another transceiver, as will be discussed in more detail below. After receiving this data, patient support apparatus 20 forwards the medical data to patient support apparatus server 138 via its network transceiver 160. In a second manner, the UWB device 250 may include its own network transceiver (e.g. transceiver 130) and transmit its data to patient support apparatus server 138 without routing it through a patient support apparatus 20. In a third manner, the UWB device 250 may transmit its data to a non-patient support apparatus UWB device 250, and that recipient non-patient support apparatus UWB device 250 may then forward the data to patient support apparatus server 138 using its network transceiver. Regardless of how the medical data gets to patient support apparatus server 138, patient support apparatus server 138 automatically forwards the data to the correct patient's record in the EMR server 136 and/or to one or more data displays.

The forwarding of data from a UWB device 250 to the correct patient record in EMR server 136 and/or to the correct display device and/or to the correct electronic device 162 takes place automatically. That is, it does not require a caregiver to take any manual steps to associate a UWB device 250 with a particular patient. Instead, these associations—as well as the automatic routing of data to the correct patient EMR record, correct display device, and/or correct electronic device 162—are carried out automatically by the UWB device 250 and patient support apparatus server 138 after the UWB device 250 is moved to a location where it can associate itself with a locator unit 142 (either directly or through another UWB device 250, as discussed more below). Once in this position, the UWB device 250 either automatically reads the unique ID of the locator unit 142 from the locator unit 142 itself, or reads the unique ID from another UWB device 250 that is already associated with the locator unit 142. A unique ID of the UWB device and the unique ID of the locator unit 142 are then forwarded to patient support apparatus server 138 via a network transceiver onboard the UWB device 250 (or another UWB device 250 with which it is in communication). Patient support apparatus server 138 uses the ID of the locator unit 142 and the ID of the UWB device 250 to determine the room and/or bay in which that particular UWB device 250 is currently located. From that room and/or bay, patient support apparatus server 138 may then determine the patient assigned to that particular room and/or bay number (by consulting ADT server 170). In addition, patient support apparatus server 138 thereafter automatically forwards data from that particular patient support apparatus 20 to the correct patient record in the EMR server 136, to the correct display device, and/or to the correct electronic device 162. As will be discussed in greater detail, this all happens automatically once the UWB device 250 is moved within a threshold proximity to a locator unit 142 and/or another UWB device 250 that is already associated with a locator unit 142.

In an alternative embodiment, patient support apparatus server 138 is adapted to forward the patient ID associated with a particular UWB device 250 to the UWB device 250 itself. In this embodiment, the UWB device 250 itself can forward its data directly to EMR server 136 and bypass patient support apparatus server 138. In other words, once the UWB device 250 is informed of the patient to which it is associated, it is able to forward its data to EMR server 136 without passing through patient support apparatus server 138. In such embodiments, patient support apparatus server 138 may also, or alternatively, forward a corresponding display device ID and/or electronic device ID to the UWB device 250 so that the UWB device 250 can communicate its data directly to the appropriate display device and/or electronic device 162 without using patient support apparatus server 138 as a communication intermediary.

It will be understood that the architecture and content of local area network 134 will vary from healthcare facility to healthcare facility, and that the example shown in FIG. 6 is merely one example of the type of network a healthcare facility may be employ. Typically, one or more additional servers 172 will be hosted on network 134 and one or more of them may be adapted to communicate with patient support apparatus server 138. Local area network 134 will also typically allow one or more conventional electronic devices 162 to access the local area network 134 via wireless access points 132. Such electronic devices 162 include, but are not limited to, smart phones, tablet computers, portable laptops, desktop computers, smart televisions, and other types of electronic devices that include a WiFi and/or Ethernet capability and that are provided with the proper credentials (e.g. SSID, password, etc.) to access network 134 (and, in at least some situations, patient support apparatus server 138).

Linked locator units 142 (FIG. 6) are adapted to wirelessly receive signals from patient support apparatus 20 and deliver the signals to communications outlet 144 in a manner that matches the way the signals would otherwise be delivered to communications outlet 144 if a conventional nurse call cable 156 were connected directly between patient support apparatus 20 and communications outlet 144. Linked locator units 142 are also adapted to transmit signals received from communications outlet 144 to patient support apparatus 20 via a BT transceiver 198 and/or a UWB transceiver 186 (FIG. 7). Thus, patient support apparatus 20 and linked locator unit 142 cooperate to send signals to, and receive signals from, communications outlet 144 in a manner that is transparent to communications outlet 144 such that outlet 144 cannot detect whether it is in communication with patient support apparatus 20 via a wired connection or it is in communication with patient support apparatus 20 via a wireless connection between patient support apparatus 20 and linked locator unit 142 (the latter of which is in wired communication with outlet 144). In this manner, a healthcare facility can utilize the wireless communication abilities of one or more patient support apparatuses 20 without having to make any changes to their existing communication outlets 144.

As noted, in addition to sending signals received from patient support apparatus 20 to communications outlet 144, linked locator units 142 are also adapted to forward signals received from communications outlet 144 to patient support apparatus 20. Linked locator units 142 are therefore adapted to provide bidirectional communication between patient support apparatus 20 and communications outlet 144. This bidirectional communication includes, but is not limited to, communicating command signals from any of controls 50 to corresponding room devices 146, 148, and/or 150 and communicating audio signals between a person supported on patient support apparatus 20 and a caregiver positioned remotely from patient support apparatus 20. The audio signals received by locator units 142 from a microphone on patient support apparatus 20 are forwarded to communications outlet 144 (for forwarding to nurse call system 152), and the audio signals of a remotely positioned nurse that are received at communications outlet 144 (from nurse call system 152) are forwarded to a speaker onboard patient support apparatus 20.

Nurse call cable 156, in some embodiments, includes a conventional 37 pin connector on each end, one of which is adapted to be inserted into outlet 144 and the other one of which is adapted to be inserted into locator unit 142. Such 37 pin connections are one of the most common types of connectors found on existing walls of medical facilities for making connections to the nurse call system 152 and room devices 146, 148, and 150. Linked locator unit 142 and nurse call cable 156 are therefore configured to mate with one of the most common type of communication outlets 144 used in medical facilities. Such 37 pin connectors, however, are not the only type of connectors, and it will be understood that linked locator units 142 can utilize different types of connectors that are adapted to electrically couple to different types of nurse call cables 156 and/or different types of communication outlets 144. One example of such an alternative communications outlet 144 and cable 156 is disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION, the complete disclosure of which is incorporated herein by reference. Still other types of communication outlets 144 and corresponding connectors may be utilized.

Linked locator unit 142 (FIG. 6) also includes an electrical cord 176 having a plug positioned at a far end that is adapted to be inserted into a conventional electrical outlet 178. Electrical cord 176 enables linked locator unit 142 to receive power from the mains electrical supply via outlet 178. It will be appreciated that, in some embodiments, linked locator unit 142 is battery operated and cord 176 may be omitted. In still other embodiments, linked locator unit 142 may be both battery operated and include cord 176 so that in the event of a power failure, battery power supplies power to linked locator unit 142, and/or in the event of a battery failure, electrical power is received through outlet 178. Unlinked locator units 142 may also include a battery, electrical cord, or both.

In some embodiments, locator units 142 (linked and/or unlinked) include a video port/transceiver 200 that is adapted to communicate with a display device 182. In some embodiments, video port/transceiver 200 is adapted to receive a display cable 180 (FIG. 6), while in other embodiments, video port/transceiver 200 may include a wireless transceiver that is adapted to wirelessly transmit display signals to a display device 182. The display cable 180 is adapted to couple to locator unit 142 at one end and a display device 182 at its opposite send. Locator unit 142 is configured to use cable 180 to send data to display device 182 that is to be displayed thereon. Such data may include data from any of the UWB devices 250, including, but not limited to, patient temperature management device 46, patient support apparatus 20, and/or locator unit 142. Cable 180 may be a High-Definition Multimedia Interface (HDMI) cable, a Video Graphics Array (VGA) cable, a DisplayPort (DP) cable, a plurality of Radio Corporation of America (RCA) cables, a Digital Visual Interface (DVI) cable, and/or another type of cable. When adapted to receive a cable, video port/transceiver 200 is configured to include a complementary type of connector that mates with a connector on an end of cable 180. Patient support apparatus 20 may also, or alternatively, be configured to communicate directly with certain display devices without using locator unit 142 as a communication intermediary.

In addition to any of the structures and functions described herein, locator units 142 are configured to communicate location data to an associated UWB device 250 that enables the UWB device 250 and/or patient support apparatus server 138 to determine the location of UWB device 250 within the healthcare facility. In general, such location determination is carried out by the UWB device 250 analyzing UWB signals communicated between itself and locator unit 142 in order for it to determine its position relative to locator unit 142. After determining its relative position to locator unit 142, the UWB device 250 is configured to determine if its relative position meets an association threshold. If it does, the UWB device 250 uses the receipt of a unique wall identifier (ID) 190 (FIG. 7) from locator unit 142 to determine, or have patient support apparatus server 138 determine, its absolute position within the healthcare facility. The location of each locator unit 142 in the healthcare facility is surveyed during the installation of locator units 142, and the unique IDs 190 of each locator unit 142 are also recorded during the installation of locator units 142. This surveying information and corresponding ID information may be stored in patient support apparatus server 138 and/or onboard any one or more of the different types of UWB devices 250, thereby enabling the UWB device 250 and/or patient support apparatus server 138 to determine the location of a UWB device 250 within the healthcare facility once its relative position to an identified locator unit 142 is known.

If the location of a UWB device 250 is determined remotely, the UWB device 250 sends its relative position information and the ID 190 of the locator unit 142 (and its own unique ID, such as patient support apparatus ID 184 (FIG. 7)) to server 138. Server 138 includes a table of all of the locations of the locator units 142 (which, as noted, is generated via a surveying operation during the installation of locator units 142), and it uses that table to correlate the UWB device IDs and the locator unit IDs 190 it receives to specific locations within the healthcare facility. Thus, if a UWB device 250 (with a particular ID) sends a locator unit ID 190 that corresponds to room 430, server 138 determines that that particular UWB device 250 is currently located in room 430. Generally speaking, and as will be discussed in greater detail below, the location of a UWB device 250 is deemed to correspond to the location of a locator unit 142 it is currently associated with (either directly, or through another UWB device 250 that it is associated with and that is currently associated with that locator unit 142). If a UWB device 250 is not directly or indirectly associated with a locator unit 142, its location may be considered indeterminate by patient support apparatus server 138.

In some embodiments of UWB devices 250 and locator units 142, the determination of the relative location of a UWB device 250 to a locator unit 142 is carried out solely using ultra-wideband communication between the UWB device 250 and the locator unit 142. Alternatively, in some embodiments, the UWB device 250 solely uses short range infrared communications with locator unit 142 to determine its relative location, wherein such short range infrared communications are only possible when the UWB device 250 is positioned within a close proximity to the locator unit 142 (e.g. in the range of about 1-3 meters). In these latter embodiments, the UWB device 250 may report to server 138 that its location coincides with that of the nearby locator unit 142 when it is able to successfully communicate with the nearby locator unit 142 using these short range infrared communications, and to not report any location data when it is not able to successfully communicate with the nearby locator unit 142. Still further, in some embodiments, UWB devices 250 and locator units 142 may communicate with each other using both infrared and ultra-wideband communications. Further details regarding the use of short range infrared communications for location determination are described in commonly assigned U.S. Pat. No. 9,999,375 issued Jun. 19, 2018, to inventors Michael Hayes et al. and entitled LOCATION DETECTION SYSTEMS AND METHODS, the complete disclosure of which is incorporated herein by reference.

In some embodiments, locator units 142 and/or patient support apparatuses 20 may be constructed to include any or all of the functionality of the wireless headwall units and/or patient support apparatuses disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION; in commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION; and/or in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosures of all of which are incorporated herein by reference.

Still further, in some embodiments, locator units 142 and/or patient support apparatuses 20 may be constructed to include any of the features and/or functions of the headwall units 144a and/or patient support apparatuses disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference. In some embodiments, locator units 142 may be the same as, or include any of the same functionality as, the Secure® Connect™ wireless wall unit sold by Stryker Corporation of Kalamazoo, Michigan, and described in the Secure® Connect™ Operations/Maintenance Manual (document: 25212-009-201 Rev. AC.0) published in November of 2021 by Stryker Corporation of Kalamazoo, Michigan, the complete disclosure of which is incorporated herein by reference.

FIG. 7 depicts a block diagram of a linked locator unit 142 and a plurality of UWB devices 250, including a patient support apparatus 20, a patient temperature management device 46, and a generic UWB device 250. As will be discussed in greater detail below, the UWB devices 250 are configured to automatically determine whether to associate themselves with locator unit 142 and/or patient support apparatus 20 using position information they gather from the respective UWB communications. In some embodiments, if a UWB device 250 associates itself with a locator unit 142 and/or another UWB device 250 that is associated with a locator unit 142, the UWB device 250 may send data to one or more of display devices 182, and/or it may forward data to patient support apparatus server 138 and/or EMR server 136.

Locator units 142 (both linked and unlinked) include an ultra-wideband transceiver 186, a locator unit controller 188, and a unit ID 190 (FIG. 7). Linked locator units 142 further include configuration circuitry 192, a television controller 194, a headwall interface 196, and a Bluetooth transceiver 198 (FIG. 7). In some embodiments, linked and/or unlinked locator units 142 may further include a video port/transceiver 200 and/or an infrared transceiver 202. Bluetooth transceiver 198 is adapted to communicate with a Bluetooth transceiver 210 onboard patient support apparatus 20 using RF waves in accordance with the conventional Bluetooth standard (e.g. IEEE 802.14.1 and/or the standard maintained by the Bluetooth Special Interest Group (SIG) of Kirkland, Washington, USA). In some embodiments, transceivers 198 and 210 utilize Bluetooth Low Energy communications. In some embodiments, locator unit 142 may also include a network transceiver, such as a WiFi transceiver, that enables the locator unit 142 to communicate via access points 132 with patient support apparatus server 138 and/or other servers on network 134.

Ultra-wideband transceiver 186 is adapted to communicate with one or more ultra-wideband transceivers 212 positioned onboard patient support apparatus 20 and/or with one or more ultra-wideband transceivers positioned onboard another type of UWB device 250. Transceiver 186 is adapted to determine a distance between itself and the UWB transceivers of patient support apparatus 20 and/or other UWB devices 250. Alternatively, or additionally, transceiver 186 may be adapted to allow each UWB transceiver 212 onboard patient support apparatus 20 and/or each UWB transceiver onboard another UWB device 250 to determine its distance from transceiver 186. In some embodiments, the UWB transceivers 186, 212 (and other UWB transceivers) use time of flight (TOF) computations to determine these distances. In other embodiments, the UWB transceivers of UWB devices 250 and locator units 142 may utilize other techniques for determining their distances from each other, either in addition to, or in lieu of, TOF computations. In some embodiments, the UWB transceivers of devices 250 and locator units 142 may also determine an angle between themselves using angular information derived from antenna arrays positions onboard the UWB transceivers, or by using other techniques. In some embodiments, as will be discussed more below, three or more UWB transceivers 212 are positioned onboard patient support apparatus 20 and determine the relative position of UWB transceiver 186 by measuring the time difference of arrival at each transceiver 212 of a UWB signal sent from UWB transceiver 186. These time differences of arrival are used with the position and orientation of each transceiver 212 onboard patient support apparatus 20, which are known and stored in an onboard memory, to determine the position and orientation of patient support apparatus 20 with respect to the locator unit(s) 142. Such position and/or orientation determinations may be carried out using conventional triangulation and/or trilateration techniques using the distance measurements and the known positions and orientations of UWB transceivers 212 (and in some cases the angle measurements may also be used as well).

In some embodiments, the non-patient support apparatus UWB devices 250 may also include multiple UWB transceivers, similar to patient support apparatus 20. However, in some embodiments, the non-patient support apparatus UWB devices 250 may include only a single UWB transceiver. Still further, in some embodiments, different UWB devices 250 may include different numbers of UWB transceivers.

In some embodiments, the UWB transceivers of locator units 142 and UWB devices 250 (FIG. 7) are implemented as any of the Trimension™ ultra-wideband modules available from NXP Semiconductors of Austin, Texas. These modules include, but are not limited to, the Trimension™ UWB modules ASMOP1BO0N1, ASMOP1CO0R1, and/or the ASMOP1CO0A1, that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement the UWB transceivers of UWB devices 250 and locator units 142.

Locator unit controller 188 is adapted to control the operation of the components of locator unit 142, including transceivers 186, 198, configuration circuitry 192, TV controller 194, headwall interface 196, video port 200, and, if included, IR transceiver 202 (FIG. 7). When infrared transceiver 202 is included, it may be included to provide backwards compatibility to patient support apparatuses 20 that are not equipped with a UWB transceiver 212. That is, some healthcare facilities may include one or more patient support apparatuses that are not equipped with a UWB transceiver 212, but that do include an IR transceiver that is adapted to communicate with IR transceiver 202. When linked locator unit 142 includes IR transceiver 202, it is able to communicate its unit ID 190 to such patient support apparatuses via IR transceiver 202, which is a short range transceiver that is configured to only communicate with an adjacent patient support apparatus when the patient support apparatus is nearby (e.g. without about 1 to 3 meters or so). Such an adjacent patient support apparatus then communicates the received locator unit ID 190 along with its own unique ID 184 to server 138 which, as noted previously, is able to correlate the locator unit ID 190 to a particular location with the healthcare facility. In this manner, server 138 is able to use locator units 142 determine the location of versions of patient support apparatuses 20 that don't have a UWB transceiver 186, but that do have an IR transceiver.

Headwall interface 196 (FIG. 7) is adapted to change the electrical state of one or more pins that are in electrical communication with communication outlet 144 (via cable 156). Headwall interface 196 changes these electrical states in response to instructions from controller 188. For example, if the exit detection system 214 of patient support apparatus 20 detects a patient exit, a controller 216 of patient support apparatus 20 sends an exit alert signal to linked locator unit 142 and controller 188 responds by instructing headwall interface 196 to change the electrical state of at least one pin that is used to signal an exit alert (or a generic priority alert) to the nurse call system 152 via communications outlet 144. In some embodiments, headwall interface 196 may be constructed in the same manner as, and/or may include any one or of the functions as, the cable interface 88 described in commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCING, the complete disclosure of which is incorporated herein by reference. Alternatively, or additionally, headwall interface 196 may be constructed in the same manner as, and/or may include any one or more of the same functions as, the headwall interface 120 disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference. Linked locator unit 142 may also be configured to perform any of the functions of the headwall units 94 disclosed in the above-mentioned '778 patent application.

Configuration circuitry 192 and TV controller 194 may be configured to perform any of the same functions as, and/or be constructed in any of the same manners as, the configuration circuitry 132 and the TV control circuit 134, respectively, of commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which has already been incorporated herein by reference. Additionally, or alternatively, linked locator unit 142 may be configured to perform any of the functions of the headwall units 144 disclosed in the aforementioned '508 patent application.

Patient support apparatus 20 includes a controller 216, a memory 218, exit detection system 214, a microphone 220, Bluetooth transceiver 210, one or more UWB transceivers 212, display 52, network transceiver 160, and a plurality of additional components that are not shown in FIG. 7. Each UWB transceiver 212 is positioned at a known location on patient support apparatus 20. This known location information is stored in memory 218 and/or elsewhere, and may be defined with respect to any suitable common frame of reference. The known location information may include the spatial relationship between UWB transceivers 212 and/or any other components of patient support apparatus 20. For example, in some embodiments, the known location information includes the spatial relationship not only between UWB transceivers 212, but also the spatial relationships between UWB transceivers 212 and one or more of the following: the head end 38 of patient support apparatus 20, the foot end of patient support apparatus 20, the sides of patient support apparatus 20, the floor, and/or other components and/or landmarks of patient support apparatus 20. In some embodiments, this location information is used to determine the position and/or orientation of patient support apparatus 20 with respect to one or more walls, locator units 142, another patient support apparatus 20, and/or other objects or structures within the healthcare facility.

Controller 216, as well as controller 188, may take on a variety of different forms. In the illustrated embodiment, each of these controllers is implemented as a conventional microcontroller. However, these controllers may be modified to use a variety of other types of circuits-either alone or in combination with one or more microcontrollers-such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controllers 188 and 216 when carrying out the functions described herein, as well as the data necessary for carrying out these functions, are stored in a corresponding memory that is accessible to that particular controller (e.g. memory 218 for controller 216, and a memory (not shown) for controller 188). In some embodiments, controller 216 may include and/or work with a microcontroller that is integrated into, or associated with, UWB transceiver(s) 212, and controller 188 may include and/or work with a microcontroller that is integrated into, or associated with, UWB transceiver 186.

Controller 216 utilizes transceivers 212 to determine the relative position of patient support apparatus 20 with respect to other nearby UWB devices 250, as well as any nearby locator units 142. If patient support apparatus 20 is positioned within range of a locator unit 142 and/or another UWB device 250, its UWB transceivers 212 communicate with the UWB transceiver(s) of the locator unit 142 and/or other UWB device 250 and exchange signals that enable them to determine the distance between themselves, the relative position of each other, and/or the orientation with respect to each other. As was noted, in some embodiments, patient support apparatus 20 and/or other UWB devices 250 may include more than one UWB transceiver, in which case the controller of the respective UWB device 250 determines the distance between, and/or relative position of, each one of the multiple UWB transceivers with respect to the other UWB transceivers that are in communication range.

In some embodiments, UWB transceivers 186, 212 (FIG. 7) may also be configured to determine an angular relationship between themselves. The distance (and angle information) in at least some embodiments is calculated by UWB transceiver 212 and controller 216 of patient support apparatus 20. In other embodiments, UWB transceiver 186 and controller 188 may calculate the distance (and angle information) and forward the results of this calculation to patient support apparatus 20 (either via UWB transceiver 186 or BT transceiver 198). In either situation, patient support apparatus controller 216 is informed of the distances (and, in some embodiments, as noted, the angle information) between transceivers 212 and 186. These distances and orientations are then used to calculate a relative position of patient support apparatus 20 to the locator unit 142 in a common frame of reference that, as will be discussed in greater detail below, may be defined in a fixed relationship to the patient support apparatus 20 or in a fixed relationship to the locator unit 142.

The location of patient support apparatus 20 relative to locator units 142 is repetitively determined by an exchange of communication signals between UWB transceivers 186 and 212. This exchange is initiated by an interrogation signal that may be sent by the UWB transceivers 186 of the locator unit 142, and/or it may be sent by the UWB transceivers 212 of the patient support apparatuses 20. The trigger for sending these interrogation signals (from either source) may simply be the passage of a predefined interval of time, in at least some embodiments. That is, in some embodiments, patient support apparatus 20 and/or locator units 142 may be configured to periodically send out an interrogation signal that will be responded to by any UWB transceivers 186 or 212 that are positioned within range of that signal. In those embodiments where patient support apparatuses 20 are configured to send out such an interrogation signal, the time intervals between the interrogation signals may be varied depending upon the location and/or other status of the patient support apparatus 20. For example, in some embodiments, patient support apparatuses 20 may be configured to send out the interrogation signals with longer timer intervals between them when the patient support apparatus is stationary, and to send out the interrogation signals with shorter time intervals between them when the patient support apparatus 20 is in motion. The interrogation signals, in some embodiments, that are sent out by patient support apparatus 20 are also used to interrogate not only UWB transceiver 186 of locator unit 142, but also any UWB-equipped devices that are positioned within range of the UWB transceivers 212. Such UWB-equipped devices may include, for example, a patient temperature management device 46, one or more hoses 56 with UWB transceivers, one or more thermal wraps 54 with UWB transceivers, and/or one or more other types of medical devices.

The measured distances (and/or angular information) between the UWB transceivers 186 of locator unit 142 and/or the UWB transceivers of the other in-range UWB devices 250 may be generated using Angle of Arrival (AoA) information, Time of Flight (TOF) information, Channel State Information, Time Difference of Arrival (TDoA) information, Two-Way Ranging (TWR) ranging information, and/or other information that is generated from the communication between the UWB transceivers. In some embodiments, each UWB transceiver includes an array of antennas that are used to generate distance and/or angular information with respect to the UWB transceiver in which it is in communication. Still further, in some embodiments, the UWB transceivers include one or more of their own microcontrollers, and the location of the other UWB transceiver(s) on other devices may be determined by these internal microcontrollers without utilizing the main controller (e.g. controller 216 of patient support apparatus 20) of the UWB device 250.

Patient support apparatus 20 also includes, in at least some embodiments, a microphone 220 (FIG. 7) that is used to detect the voice of the patient when the patient wants to speak to a remotely positioned nurse. The patient's voice is converted to audio signals by microphone 220 and controller 216 is adapted to forward these audio signals to an adjacent communications outlet 144 positioned in wall 154 (FIG. 6). When a cable 156 is coupled between patient support apparatus 20 and outlet 144, controller 216 forwards these audio signals to outlet 144 via the cable. When no such cable 156 extends between patient support apparatus 20 and outlet 144, controller 216 wirelessly forwards these audio signals to the locator unit 142 that it is currently associated with (using transceiver 210, or in some embodiments, transceiver 212) and controller 188 of locator unit 142 forwards these audio signals to outlet 144. As was noted, outlet 144 is in electrical communication with a conventional nurse call system 152 that is adapted to route the audio signals to the correct nurse's station 174, and/or other location. In some embodiments, microphone 220 acts as both a microphone and a speaker. In other embodiments, a separate speaker may be included in order to communicate the voice signals received from the remotely positioned nurse. In some embodiments, the audio communication between patient support apparatus 20 and communications outlet 144 is carried out in any of the manners, and/or includes any of the structures, disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

After the installation of locator units 142 in a particular healthcare facility, the location of each locator unit 142 within that facility is recorded. In some embodiments, the coordinates of the locations of locator units 142 are recorded in a common frame of reference (or converted to a common frame of reference after recordation). Such coordinates may be three dimensional (i.e. include a height components), or they may be two dimensional (no height component). In other embodiments, a more generalized location of one or more locator units 142 is determined, rather than the precise coordinates of the locator units 142. In still other embodiments, the locations of one or more locator units 142 are determined both generally and more precisely. The generalized location of the locator units 142 may include an indication of the room, bay, area, hallway, portion of a hallway, wing, maintenance area, etc. that the locator unit 142 is positioned in. The specific location of the locator units 142, as noted, may include an X, Y, and Z coordinate within a common frame of reference.

Regardless of how the location of each locator unit 142 is initially determined after they are installed in a healthcare facility (e.g. whether their coordinates are determined or a more generalized location is determined), the locations of all of the locator units 142, as well as their unique IDs 190, are stored in a memory accessible to server 138. Server 138 then uses this location data and ID data to determine the location of one or more UWB devices 250. Alternatively, or additionally, the location data and ID data are forwarded to patient support apparatuses 20 and/or other UWB devices 250 for storage in their onboard memories and for use in determining their own locations. In some embodiments, the location of each locator unit 142 (whether specific and/or general) may also, or alternatively, be stored in a memory within that particular locator unit 142 and shared with the UWB devices 250 it communicates with (e.g. patient support apparatuses 20). In some other embodiments, the location of each locator unit 142 may be stored in multiple locations.

It will be appreciated that patient support apparatuses 20 are configured to communicate with locator units 142 regardless of the orientation of the patient support apparatus 20. That is, the UWB transceivers 186 and 212 are radio frequency transceivers that do not rely on line of sight communication, unlike the IR transceiver 202 (if present). Thus, the patient support apparatuses 20 do not have to be pointed in any particular direction with respect to the locator units in order for transceivers 186 and 212 to communicate. This differs from some prior art systems that use IR communication between the patient support apparatuses 20 and the locator units and that require the IR transceiver onboard the patient support apparatus to be aimed toward the locator unit in order for communication to be established. It will also be understood that locator units 142 can be positioned on wall, columns, ceilings, or any other fixed structures within the healthcare facility.

As noted, patient support apparatus 20 is also configured to use UWB transceivers 212 to determine the position of various other UWB devices 250 relative to patient support apparatus 20, such as one or more patient temperature management devices 46 and/or other types of UWB devices 250. As will be discussed in greater detail below, controller 216 uses UWB transceivers 212 to determine the relative position of these UWB devices 250 by communicating with one or more respective UWB transceivers that are either built into those other UWB devices 250 or attached to a tag that is affixed to those devices 250. Such UWB transceivers operate in the same manner as UWB transceivers 212 and/or UWB transceiver 186 of locator units 142. And, as will also be discussed in greater detail below, controller 216 of patient support apparatus 20 uses the relative position information to determine how it will interact with these UWB devices 250, including whether to associate itself with these devices 250, whether to display data from these devices 250, and/or whether to send data from one or more of these devices 250 to patient support apparatus server 138 and/or EMR server 136.

Display device 182 includes a display 240 and a display controller 242. Display device 182 may take on a variety of different forms. In some embodiments, display device 182 may be a conventional smart phone, laptop computer, tablet computer, smart TV and/or smart monitor. Display device 182 may interact with any UWB device 250 in any of the same manners that the patient support apparatus and display devices interact with each other that are disclosed in commonly assigned U.S. patent application Ser. No. 63/306,279 filed Feb. 3, 2022, by inventors Madhu Thota et al. and entitled COMMUNICATION SYSTEM FOR PATIENT SUPPORT APPARATUS, the complete disclosure of which is incorporated herein by reference. That is, one or more UWB devices 250 may be configured to automatically associate themselves with one or more display devices 182 and forward data to be displayed thereon, as will be described in more detail below.

As shown in FIG. 7, tertiary UWB device 250 includes a UWB transceiver 252 and a unique device ID 254. It will be understood that tertiary UWB device 250 is a generic UWB device that is intended to represent a wide variety of different UWB devices 250. Accordingly, tertiary UWB device 250 of FIG. 7 may include a wide variety of different components in addition to transceiver 252 and device ID 254. In general, tertiary UWB device 250 may include, but is not limited to, any one or more of the following: exercise devices, heel care boots, IV stands and/or poles, infusion pumps, ventilators, DVT pumps, patient monitors (e.g. saturated oxygen (Sp0₂) monitors, EKG monitors, vital sign monitors, etc.), patient positioning devices (e.g. wedges, turning devices, pumps, etc.), devices used during surgery (e.g. surgical tools and instruments, surgical waste collection systems, lights, cameras, etc.), ambient sensors (e.g. air temperature, air flow, light, humidity, pressure, altitude, sound/noise, etc.), mattress 42, an incontinence pad or one or more sensors adapted to detect patient incontinence, a Holter device adapted to monitor and record a patient's heart signals, a patient ID tag or bracelet worn by the patient that identifies the patient, a caregiver tag or badge worn by a caregiver that identifies the caregiver (and/or that communicates with the badge server hosted on network 134), one or more pieces of furniture that a patient may be expected to use, and/or other types of devices. In general, UWB devices 250 include any devices whose position and association status may be determined through communications with patient support apparatus 20 and/or locator unit 142, such as any devices that are used in a medical setting for treating, diagnosing, monitoring, and/or caring for a patient.

FIG. 7 illustrates three UWB devices 250: a patient support apparatus 20, a patient temperature management device 46, and a generic UWB device 250. Each UWB device 250 may be classified as one of three different types of UWB devices: a primary UWB device 250, a secondary UWB device 250, and a tertiary UWB device 250. Each different classification of the UWB devices 250 is based upon the association algorithm 128 that the device 250 follows. In other words, a primary UWB device 250 follows a first set of rules (e.g. algorithm 128a) for automatically communicating with a locator unit 142 and/or other UWB devices 250; a secondary UWB device 250 follows a second and different set of rules (e.g. algorithm 128b) for automatically communicating with a locator unit 142 and/or other UWB devices 250; and a tertiary UWB device follows a third and different set of rules (e.g. algorithm 128c) for automatically communicating with a primary or secondary UWB device 250 (tertiary UWB devices 250 do not automatically communicate with a locator unit 142). The different sets of rules dictate how and when each UWB device 250 will associate with one or more devices having UWB communication abilities.

In at least one embodiment, each patient support apparatus 20 is a primary UWB device 250, and one or more secondary and/or tertiary UWB devices 250 are adapted to communicate with patient support apparatus 20 in the manner described herein. Each primary UWB device 250 is adapted to automatically determine its position relative to a locator unit 142 and to automatically associate itself with that locator unit 142 if the patient support apparatus 20 is positioned inside of a predefined volume of space defined with respect to the locator unit 142, or if the locator unit 142 is positioned inside of a predefined volume of space defined with respect to patient support apparatus 20. In the example shown in FIG. 6, volume of space A may be defined with respect to either patient support apparatus 20 or with respect to locator unit 142. Each primary UWB device 250 is also adapted to associate one or more secondary or tertiary UWB devices 250 with the primary UWB device 250 if the secondary and/or tertiary UWB devices 250 are positioned within a predefined volume of space defined with respect to the primary UWB device 250. In the example shown in FIG. 6, volume of space B may be defined with respect to the primary UWB device 250 (e.g. patient support apparatus 20).

Secondary UWB devices 250 are configured to communicate with a primary UWB device 250 and to associate themselves with the primary UWB device 250 if a primary UWB device is within communication range and the secondary UWB device 250 is within a predefined volume of space (e.g. volume of space B) defined with respect to the primary UWB device 250. If the secondary UWB device 250 is not able to communicate with a primary UWB device 250 (e.g. because no primary UWB device 250 is within range), or if the secondary UWB device 250 is able to communicate with a primary UWB device 250 but the secondary UWB device 250 is positioned outside of the predefined volume of space defined with respect to the primary UWB device, the secondary UWB device 250 is configured to automatically attempt to use UWB signals to communicate with any locator unit 142 that is positioned within range of the secondary UWB device 250. If the secondary UWB device 250 is able to communicate with a locator unit 142, the secondary UWB device 250 is further configured to either automatically determine if the secondary UWB device 250 is positioned within a predefined volume of space defined with respect to the locator unit 142 (such as volume of space A in FIG. 6), or to automatically determine if the locator unit 142 is positioned within a predefined volume of space defined with respect to the secondary UWB device 250. If either of these conditions is met (the secondary UWB device 250 is positioned within a predefined volume of space defined with respect to the locator unit 142, or the locator unit 142 is positioned within a predefined volume of space defined with respect to the secondary UWB device 250), then the secondary UWB device 250 is configured to automatically associate itself with the locator unit 142.

Once a secondary UWB device 250 has associated itself with a locator unit 142, it is configured to act in a similar manner to a primary UWB device 250. That is, a secondary UWB device 250 that has associated itself with a locator unit 142 is configured to automatically associate one or more tertiary UWB devices 250 with itself if those tertiary UWB devices 250 are positioned within a predefined volume of space defined with respect to the secondary UWB device 250. The secondary UWB device 250 may further be configured to automatically and periodically re-check to see if a primary UWB device 250 has moved within a predefined volume of space (defined with respect to the secondary UWB device 250 and/or the locator unit 142). If a primary UWB device 250 has moved within the predefined volume of space, the secondary UWB device 250 may automatically disassociate itself from the locator unit 142 and switch to associating itself with the primary UWB device 250. If a primary UWB device 250 has not moved within the predefined volume of space, the secondary UWB device 250 may continue to keep its association with the nearby locator unit 142.

Tertiary UWB devices 250, as noted, are adapted to automatically attempt to associate with any primary UWB device 250 that is positioned within range of the tertiary UWB device 250. If the tertiary UWB device 250 is not able to associate with a primary UWB device 250, it is configured to automatically attempt to associate with any secondary UWB device 250 that is positioned with range of the tertiary UWB device 250 and that is associated with a locator unit 142. If it is also unable to associate with a secondary UWB device 250, it does not associate with any UWB devices 250. After a predetermined amount of time, which may be variable and which may be user configurable, the tertiary UWB device 250 will re-attempt to associate with a primary UWB device 250 (and if unable, will re-attempt to associate with a secondary UWB device 250 that is associated with a locator unit 142).

In the example shown in FIG. 7, patient support apparatus 20 is a primary UWB device 250, temperature management device 46 is a secondary UWB device, and generic UWB device 250 is a tertiary UWB device 250. In this example, patient support apparatus 20 has associated itself with locator unit 142, and both patient temperature management device 46 and UWB device 250 have associated themselves with patient support apparatus 20. In some embodiments, this association is reflected by patient support apparatus 20 sending a message to patient support apparatus server 138 indicating these associations. Patient support apparatus server 138, in turn, may indicate these associations by displaying information indicating that patient support apparatus 20, temperature management device 46, and UWB device 250 are positioned in a common location (such as the room number corresponding to the specific locator unit 142 that patient support apparatus 20 is associated with), as will be discussed in greater detail below.

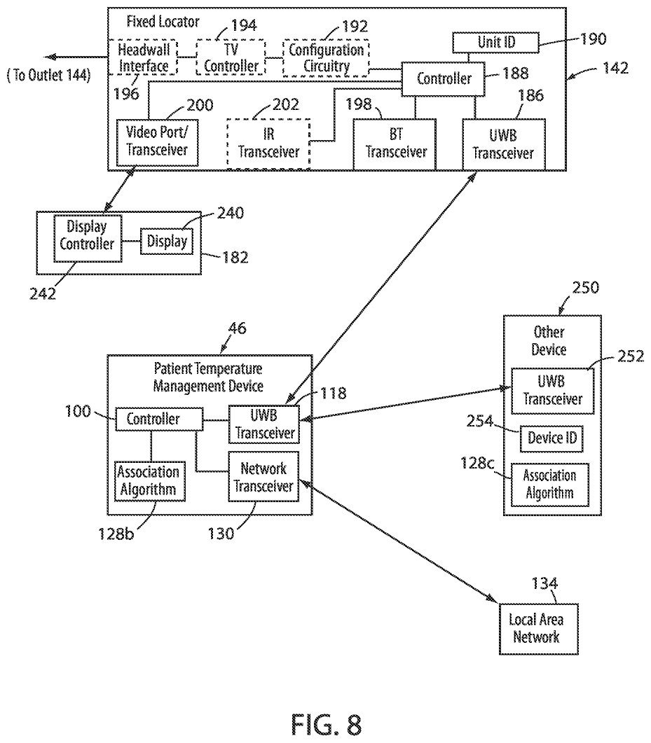

FIG. 8 illustrates another example of a plurality of UWB devices 250 that may take place in a healthcare facility. In the example of FIG. 8, there is no patient support apparatus 20 located in the vicinity of locator unit 142. Because of the lack of a patient support apparatus 20, neither temperature management device 46, which is a secondary UWB device 250, nor UWB device 250, which is a tertiary UWB device 250, are able to associate with a primary UWB device 250 (e.g. patient support apparatus 20). Also as a result of the absence of a primary UWB device 250, and because temperature management device 46 is a secondary UWB device 250, temperature management device 46 is configured to act as if it were a primary UWB device 250. That is, after confirming that no primary UWB device 250 is available for it to be associated with, temperature management device 46 is configured to attempt to associate with locator unit 142 and, if successful, to allow tertiary (and in some cases, other secondary) UWB devices 250 to associate with itself. Thus, in the situation shown in FIG. 8, temperature management device 46 has associated itself with locator unit 142 (after either determining that locator unit 142 is positioned within a predetermined volume of space (e.g. volume of space A) defined with respect to temperature management device 46, or that temperature management device 46 is positioned within a predetermined volume of space (e.g. volume of space A) defined with respect to locator unit 142). Further, after determining that tertiary UWB device 250 is positioned within a predetermined volume of space (e.g. volume of space B) defined with respect to temperature management device 46, temperature management device 46 has associated itself with tertiary UWB device 250.

As a result of the association of temperature management device 46 with locator unit 142, controller 100 of temperature management unit 46 sends a message to patient support apparatus server 138 (via network transceiver 130) that includes the unique ID 190 of the locator unit 142, as well as the unique ID 126 of the temperature management device 46. Server 138 uses this information to determine the location of temperature management device 46 within the healthcare facility. In addition, as a result of the association of temperature management device 46 with tertiary UWB device 250, temperature management device 46 may send another message to patient support apparatus server 138 that includes a unique ID 254 of the UWB device 250, as well as the unique ID 126 of the temperature management device 46. Server 138 uses this information to determine the location of tertiary UWB device 250 within the healthcare facility. That is, server 138 determines that the tertiary UWB device 250 is in the same location as the temperature management device 46.

In general, secondary UWB devices 250 may include, but are not limited to, any one or more of the following: exercise devices, heel care boots, IV stands and/or poles, infusion pumps, ventilators, DVT pumps, patient monitors (e.g. saturated oxygen (Sp0₂) monitors, EKG monitors, vital sign monitors, etc.), patient positioning devices (e.g. wedges, turning devices, pumps, etc.), devices used during surgery (e.g. surgical tools and instruments, surgical waste collection systems, lights, cameras, etc.), ambient sensors (e.g. air temperature, air flow, light, humidity, pressure, altitude, sound/noise, etc.), mattress 42, an incontinence pad or one or more sensors adapted to detect patient incontinence, a Holter device adapted to monitor and record a patient's heart signals, a patient ID tag or bracelet worn by the patient that identifies the patient, a caregiver tag or badge worn by a caregiver that identifies the caregiver (and/or that communicates with the badge server hosted on network 134), one or more pieces of furniture that a patient may be expected to use, and/or other types of devices. In general, secondary UWB devices 250 include any devices whose position and association status may be determined through communications with patient support apparatus 20 and/or locator unit 142, such as any devices that are used in a medical setting for treating, diagnosing, monitoring, and/or caring for a patient. The designation of whether a UWB device 250 is primary, secondary, or tertiary is performed through the programming of the device 250 and the algorithm it follows for association and communication, as will be discussed in further detail below.

In some embodiments, the UWB transceivers of each UWB device 250 are configured to act as UWB anchors and/or as UWB tags. In at least one embodiment, the UWB transceivers 186 of locator units 142, as well as the UWB transceivers 252 of tertiary UWB devices 250 are configured to act as UWB tags, while the UWB transceivers 212 of primary UWB devices 250 (e.g. patient support apparatuses 20) are configured to act as anchors and the UWB transceivers 118 of secondary UWB devices 250 (e.g. temperature management device 46) are configured to act as UWB anchors at certain times and to act as UWB tags at other times. It will be understood that modifications to these roles of anchors and tags can be made. For example, in some embodiments, the UWB transceivers 212 of patient support apparatus 20 may be modified to act as UWB anchors in some instances and as UWB tags in other instances. Still other modifications can be made.

In general, when a UWB transceiver of a UWB device 250 is configured to act as a UWB tag, it is configured to periodical transmit a UWB start packet, which acts as a discovery packet. The start packet requests that any UWB anchors that are within communication range to respond. If another UWB transceiver that is acting as a UWB tag happens to receive the start packet from another UWB tag, that UWB transceiver is configured to not respond to it. In other words, tags transmit start packets, but do not respond to start packets. UWB anchors, on the other hand, do not transmit start packets, but instead respond to start packets with a response packet that may be referred to as a stamp packet. Anchors therefore transmit stamp packets, but do not transmit start packets. UWB anchors also do not respond to other stamp packets that they may detect from other UWB anchors. In response to receiving a stamp packet from a UWB anchor, the UWB tags are configured to transmit an end packet back to the UWB anchor that transmitted the stamp packet.

The combination of the start, stamp, and end packet generally defines a ranging session between a UWB anchor and a UWB tag. The ranging session uses time of flight (TOF) information contained within the start, stamp, and end packet to allow the anchor and/or tag to determine a distance between the tag and the anchor. In some embodiments, the start, stamp, and/or end packet may also contain other data in their payloads that is used for other purposes besides ranging. From the ranging information, the distance between the anchor and tag is determined. These ranging sessions are repetitively carried out while a UWB anchor and UWB tag are within communication range.

In some embodiments, the time interval between ranging sessions is controlled by the UWB tag. That is, after the UWB tag sends a start packet, receives a stamp packet in response, and sends an end packet in response to the stamp packet, the UWB tag is configured to wait a defined amount of time before sending out another start packet. The defined amount of time is programmed into the UWB tag and can be varied during operation of the UWB tag. In some embodiments, as will be discussed in greater detail below, the UWB tag may be configured to change this defined amount of time (hereinafter, the “ranging interval”) based upon whether the UWB device 250 with the tag is associated with, or not associated with, a UWB device 250 having an anchor. In other embodiments, the ranging interval may be changed by a tag based upon the status of one or more of the UWB devices 250 that are involved in the ranging session. Such status may include, but is not limited to, the movement status of one or more of the UWB devices 250, the position and/or state of one or more components of the UWB devices 250, the location of the UWB device 250 within the healthcare facility and/or in relation to other UWB device(s) 250, and/or other factors.

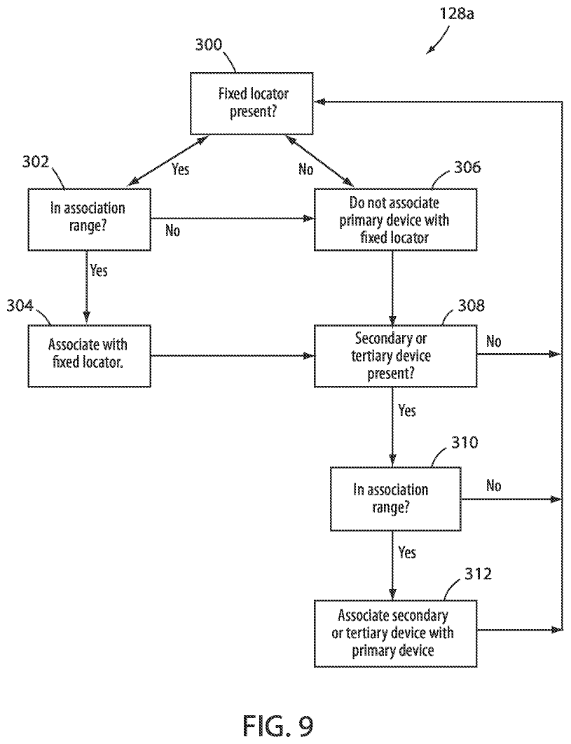

FIG. 9 illustrates one example of an association algorithm 128a that may be followed by primary UWB devices 250, such as patient support apparatuses 20. Primary association algorithm 128a begins at an initial step 300 where controller 216 of patient support apparatus 20 uses its UWB transceivers 212 to determine if a locator unit 142 is within communication range. As noted earlier, UWB transceiver 186 of locator unit 142 may be configured to act as a UWB tag, in which case it periodically transmits a start packet to any UWB device 250 within communication range. Controller 216 of patient support apparatus 20 determines at step 300 whether it has received such a start packet at step 300. If it has not, it proceeds to step 306. If it has, it proceeds to step 302. At step 302, controller 216 of patient support apparatus 20 determines if patient support apparatus 20 is within a predetermined distance of the locator unit 142. In some embodiments, controller 216 determines this distance through one or more ranging sessions between its UWB transceiver 212 and the UWB transceiver 186 of locator unit 142. Further, in some embodiments, the association distance is defined as any distance that is positioned within a volume of space, such as volume of space A (FIG. 6). As was previously noted, volume of space A may be defined with respect to patient support apparatus 20, or it may be defined with respect to locator unit 142. For either definition, controller 216 uses the distance(s) between UWB transceiver 212 and 186 to determine if patient support apparatus 20 is positioned within an association range of the locator unit 142.

In some embodiments, not only must patient support apparatus 20 be positioned within volume of space A for it to be associated with locator unit 142, but patient support apparatus 20 and locator unit 142 have to be in the same room (or on the same side of a wall to which locator unit 142 is attached) before such association can occur. In such embodiments, controller 216 and/or locator unit 142 may be constructed in accordance with, and include any of the same functionality as, the wall units 60 and patient support apparatuses 20 disclosed in commonly assigned U.S. provisional patent application Ser. No. 63/323,202 filed Mar. 24, 2022, by inventors Kirby Neihouser et al. and entitled SYSTEM FOR LOCATING PATIENT SUPPORT APPARATUSES, the complete disclosure of which is incorporated herein by reference. The patient support apparatuses 20 and wall units of this '202 application are able to automatically determine if they are positioned on the same side of a wall as each other, and the techniques for carrying out this function, as well as any of the other aspects of the system disclosed therein, may be incorporated into the patient support apparatuses 20 and locator units 142 of the present disclosure.

If controller 216 determines at step 302 (FIG. 9) that patient support apparatus 20 is positioned within an association range (e.g. volume of space A), and that patient support apparatus 20 is positioned on the same side of a wall as locator unit 142, it associates patient support apparatus 20 with that particular locator unit 142 at step 304. From step 304, controller 216 proceeds to step 308, which is discussed below.

Returning to step 300 of algorithm 128a (FIG. 9), if controller 216 does not detect a locator unit 142 within range, it proceeds to step 306. At step 306, controller 216 proceeds to dissociated itself from any previous locator unit 142 that it may have been previously associated with. If it was not previously associated with a locator unit 142, it continues to remain disassociated from any locator unit 142. From step 306, controller 216 proceeds to step 308. At step 308, controller 216 determines whether any secondary or tertiary UWB devices 250 are within communication range. Controller 216 may make this determination in a manner similar to the determination in step 300. That is, each secondary and tertiary UWB device 250 is configured, like locator unit 142, to act as a UWB tag, which means that it periodically transmits a UWB start message to any UWB transceivers that are within range. Controller 216 therefore checks at step 308 whether any of its UWB transceivers 212 have detected a start packet from a secondary or tertiary UWB device 250 at step 308. If no such start packet has been detected, controller 216 returns to step 300 and the algorithm repeats.

If controller 216 has detected a start packet from a secondary or tertiary UWB device 250, in addition to responding with a stamp packet, it proceeds to step 310 (FIG. 9). At step 310, controller 216 determines if each of the secondary and/or tertiary UWB devices 250 that it has received start packets from are currently within an association range. The association range, in some embodiments, may refer to any position that is contained with volume of space B (FIG. 6), or another predetermined volume of space defined around patient support apparatus 20. If any of the secondary or tertiary UWB devices 250 are not positioned within this range, controller 216 does not associate patient support apparatus 20 with those secondary and/or tertiary UWB devices 250. If any of the secondary or tertiary UWB devices 250 are positioned within this range, controller 216 proceeds to step 312 where it associates those device(s) 250 with patient support apparatus 20.

In some embodiments, controller 216 may be configured to require one or more additional association conditions be met before it associates itself with one or more secondary or tertiary medical devices 250 at step 310. That is, in addition to being within a range, controller 216 may be configured to require that one or more additional conditions are met before controller 216 associates patient support apparatus 20 with a particular UWB device 250. For example, in some embodiments, controller 216 may require additional criteria are met before it associates patient support apparatus 20 with a temperature management device 46. Such additional criteria may require that one or more hoses (or hose ends) attached to the temperature management device 46 are positioned within a volume of space, and/or that one or more thermal wraps adapted to be worn by a patient are positioned within a volume of space. Examples of these types of additional association criteria for associating a temperature management device with a patient support apparatus are discussed in more detail in commonly assigned U.S. provisional patent application Ser. No. 63/314,221 filed Feb. 25, 2022, by inventors Jerald Trepanier et al. and entitled COMMUNICATION SYSTEM FOR PATIENT SUPPORT APPARATUSES AND TEMPERATURE MANAGEMENT DEVICES, the complete disclosure of which is incorporated herein by reference. Other additional association criteria may also be required by controller 216 before it associates a UWB device 250 with patient support apparatus 20.

After associating patient support apparatus 20 with one or more secondary or tertiary WUB devices 250, controller 216 returns to step 300 and repeats algorithm 128a. Each time it repeats algorithm 128a, controller 216 automatically disassociates any UWB device 250 that is not within association range (and/or that doesn't meet any additional association criteria), as determined at step 310. In other words, controller 216 repetitively executes algorithm 128a to determine if any associated UWB devices 250 are currently still within range (and meet any additional criteria), as well repetitively checks to see if any new UWB devices 250 have moved within association range (and meet any additional criteria). If any previously associated UWB devices 250 have moved out of association range or otherwise stopped meeting any additional association criteria, controller 216 disassociates those devices. If any previously unassociated UWB devices 250 move into association range and meet any additional association criteria, controller 216 automatically associates those UWB devices 250 with patient support apparatus 20. In this manner, controller 216 dynamically, and automatically, updates what UWB devices 250 is it associated with and disassociated from. The repetition of algorithm 128a may occur multiple times a second, or at a slower frequency.

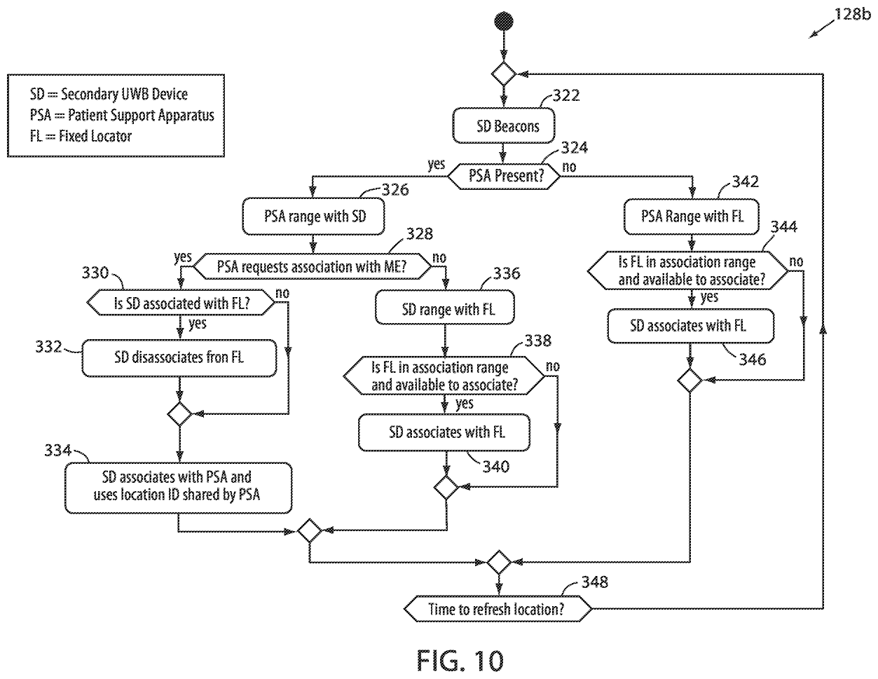

FIG. 10 illustrates one example of an association algorithm 128b that may be followed by one or more secondary UWB devices 250, such as temperature management device 46 or any other desired UWB device 250. Secondary association algorithm 128b begins at an initial step where the controller of the secondary UWB device (hereinafter the “secondary controller”) sends out a start packet from its UWB transceiver (e.g. UWB transceiver 118) at step 322. The start packet is sent out to any UWB devices 250 that are positioned within communication range. At step 324, the secondary controller determines if any stamp packets have been received from a patient support apparatus 20 in response to the start packet sent at step 322. If no stamp packet has been received from a patient support apparatus 20, the secondary controller concludes that no patient support apparatus 20 is within range and proceeds to step 342. If a stamp packet has been received from a patient support apparatus 20, the secondary controller proceeds to step 326 where it continues its ranging session with the patient support apparatus 20. That is, at step 326, the secondary controller determines the distance between the secondary UWB device 250 and the patient support apparatus 20 with which it is in communication. If the patient support apparatus 20 is within range (e.g. the secondary UWB device 250 is positioned within volume of space B), the secondary controller waits to see if the patient support apparatus 20 requests an association with the secondary UWB device 250. If no such request arrives, the secondary controller proceeds to step 336. If such a request arrives, the secondary controller proceeds to step 330.

At step 330 (FIG. 10), the secondary controller determines whether it is currently associated with a locator unit 142 or not. If it is, the secondary controller proceeds to step 332 where it disassociates itself from the locator unit 142. If it is not, the secondary controller skips step 334 and proceeds to step 334. At step 334, the secondary controller associates itself with the patient support apparatus 20. In some embodiments, the process of associating a UWB device 250 with a patient support apparatus 20 includes the patient support apparatus 20 sending the locator ID 190 (of the locator unit 190 with which it is associated, if any) to the associated UWB device 250. The associated UWB device 250, in some embodiments, may use this locator ID 190 to determine its location within the healthcare facility and/or forward this locator ID 190 to a server on the network 134 (e.g. server 138) so that the server can determine the current location of the UWB device 250.

From step 334, the secondary controller proceeds to step 348 where it proceeds to re-start algorithm 128b if sufficient time has passed. That is, the secondary controller is configured to repeat algorithm 128b at a certain frequency, and at step 348 the secondary controller determines if the time corresponding to the certain frequency has passed by or not. If it has not, it awaits until the required time has passed before returning to step 322. If it has, it proceeds immediately to step 322. As with algorithm 128a, algorithm 128b may be repeated at a variety of different frequencies, including multiple times a second, or at less frequent intervals.

If the secondary controller does not receive an association request from the patient support apparatus 20 at step 328 (FIG. 10), the secondary controller proceeds to step 336. At step 336, the secondary controller switches its UWB transceiver (e.g. UWB transceiver 118) from the tag mode of operation to the anchor mode of operation. When operating in the tag mode, the UWB transceiver listens for start packets and, if it detects one, it responds with a stamp packet, as discussed previously. Thus, at step 336, the secondary controller determines if any start packets have been received from a locator unit 142. If a start packet has been received, the secondary controller responds with a stamp packet and carries out a ranging session with the locator unit 142 and then proceeds to step 338. If no start packet has been received, the secondary controller proceeds to step 348 (not shown in FIG. 10).

At step 338 (FIG. 10), the secondary controller determines if the locator unit 142 is positioned within an association range of the secondary UWB device 250. As discussed previously, this step may involve the secondary controller determining the distance to the locator unit 142 by carrying out one or more ranging sessions between its UWB transceiver and the UWB transceiver 186 of locator unit 142. In some embodiments, the association distance is defined as any distance that is positioned within a volume of space, such as volume of space A (FIG. 6). Volume of space A may be defined with respect to the secondary UWB device 250 that the secondary controller is part of, or it may be defined with respect to locator unit 142. For either definition, the secondary controller uses the distance(s) between the UWB transceivers to determine if the secondary UWB device is positioned within an association range of the locator unit 142. If it is not, the secondary controller proceeds to step 348. If it is, the secondary controller proceeds to step 340 where it associates itself with the locator unit 142. After step 340, the secondary controller proceeds to step 348.

If the secondary controller does not detect a patient support apparatus 20 within UWB communication range at step 324, the secondary controller proceeds through steps 342, 344, and/or 346, as shown in FIG. 10. Steps 342, 344, and 346 are the same as steps 336, 338, and 340, respectively, which have been previously described and need not be described again. After completing steps 342, 344, and/or 346, the secondary controller proceeds to step 348 where it proceeds to re-start algorithm 128b at the appropriate interval.

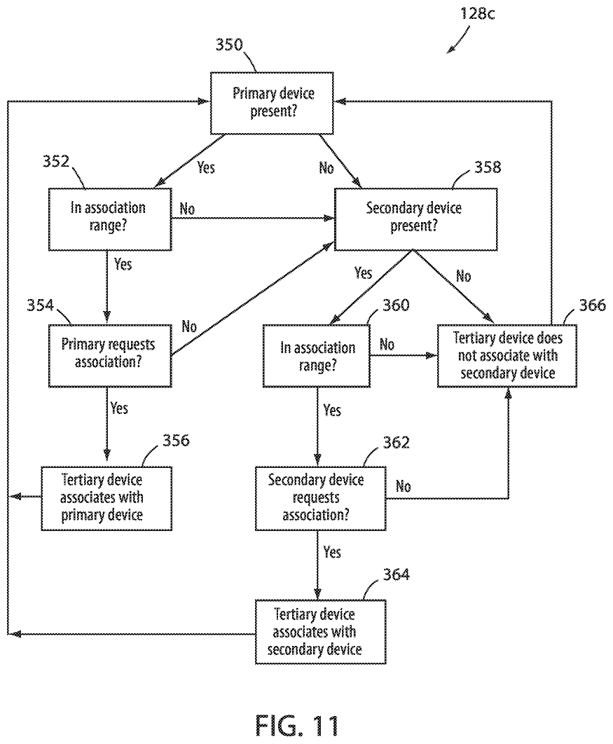

FIG. 11 illustrates one example of an association algorithm 128c that may be followed by one or more tertiary UWB devices 250. Tertiary association algorithm 128c begins at an initial step 350 where the controller of the tertiary device (hereinafter the “tertiary controller) uses its UWB transceiver(s) to determine if a primary UWB device 250 is within communication range. As noted earlier, the UWB transceiver(s) of the tertiary UWB device 250 may be configured to act as UWB tags, in which case the UWB transceiver(s) periodically transmits a start packet to any UWB device 250 within communication range and waits for a stamp packet to be transmitted in response. The tertiary controller determines at step 350 whether it has received a stamp packet in response to start packet. Specifically, it determines at step 350 if it has received a stamp packet from a primary UWB device 250, such as patient support apparatus 20. If it has not, it proceeds to step 358. If it has, it proceeds to step 352.

At step 352, the tertiary controller determines if the tertiary UWB device 250 is within a predetermined distance of the primary UWB device 250. In some embodiments, the tertiary controller determines this distance through one or more ranging sessions between its UWB transceiver and the UWB transceiver of the primary UWB device 250 (e.g. UWB transceiver(s) 212 of patient support apparatus 20). Further, in some embodiments, the association distance is defined as any distance that is positioned within a volume of space, such as volume of space B (FIG. 6) and/or another volume of space. The volume of space may be defined with respect to the primary UWB device 250, or it may be defined with respect to the tertiary UWB device 250. For either definition, the tertiary controller uses the distance(s) between its UWB transceiver(s) and the primary UWB device's UWB transceivers to determine if the primary UWB device 250 and the tertiary UWB device 250 are positioned within an association range of each other.

If the tertiary controller of the tertiary UWB device 250 determines at step 352 that it is within association range at step 354, it proceeds to step 354 where it waits to see if the primary UWB device 250 will send an association request. If such an association request is not received, the tertiary controller proceeds to step 358. If such an association request is received, the tertiary controller proceeds to step 356 where it associates itself with the primary UWB device 250. From step 356, the tertiary controller returns to step 350 and re-starts algorithm 128c at the configured frequency (e.g. multiple times a second, or a slower frequency).

When the tertiary controller moves to step 358 (either from step 350 or step 352), it determines if a secondary UWB device 250 is present or not. The tertiary controller carries out step 358 by

determining if any stamp packets have been received from a secondary UWB device 250 in response to the start packet sent at step 350. If no stamp packet has been received from a secondary UWB device 250, the tertiary controller concludes that no secondary UWB device 250 is within range and proceeds to step 366. If a stamp packet has been received from a secondary UWB device 250, the tertiary controller proceeds to step 360 where it continues its ranging session with the secondary UWB device 250. That is, at step 360, the tertiary controller determines the distance between the tertiary UWB device 250 and the secondary UWB device 250 with which it is in communication. If the secondary UWB device 250 is within range (e.g. both the secondary UWB device 250 and the tertiary UWB device 250 are positioned within a common volume of space defined with respect to either the secondary UWB device 250 or with respect to the tertiary UWB device 250), the tertiary controller waits to see at step 362 if the secondary UWB device 250 will request an association with the tertiary UWB device 250. If no such request arrives, the tertiary controller proceeds to step 366. If such a request arrives, the tertiary controller proceeds to step 364 where it associates itself with the secondary UWB device 250.

When the tertiary controller executes step 366 (FIG. 11), it disassociates itself with any secondary device that it may have previously been associated with. If the tertiary UWB device 250 was not previously associated with a secondary UWB device 250, it continues to remain disassociated with any secondary UWB devices 250. After completing step 366, the tertiary controller returns to step 350 and repeats algorithm 128c at the configured frequency.

It will be understood that substantial modifications to any one or more of algorithms 128a-c shown in FIGS. 9-11 may be made without departing from the spirit of the present disclosure. For example, in some embodiments, algorithm 128b is modified so that, if the UWB transceiver onboard the secondary UWB device 250 is acting in the anchor mode, it automatically switches back to acting in the tag mode whenever it repeats step 322. As noted, after completing step 322, it may switch its UWB transceiver back to the anchor mode whenever it proceeds to step 342 or 336.

Another modification that can be made to algorithm 128b is the incorporation of one or more steps from algorithm 128a into algorithm 128b. For example, in some embodiments, after the secondary controller completes step 340 or step 346 (associates with a locator unit 142) of algorithm 128b, the secondary controller may be configured to carry out steps 308, 310, and 312 from algorithm 128a. These steps enable other secondary or tertiary UWB devices 250 to associate themselves with the secondary UWB device 250 carrying out algorithm 128b if that secondary UWB device 250 is not associated with a primary UWB device (e.g. patient support apparatus 20). In other words, once the secondary UWB device 250 of algorithm 128b determines that there is no primary UWB device 250 to associate with, it may take on the role of the primary UWB device 250 by executing steps 308, 310, and 312 of algorithm 128a. This assumption of the role of a primary UWB device 250 may continue for as long as the secondary UWB device 250 remains unassociated with a primary UWB device 250.

Another modification that may be made to the algorithms 128a-c disclosed herein is the addition of a step between steps 310 and 312 of algorithm 128a. During this additional step, the controller of the primary UWB device 250 may be configured to send out a request to associate to the secondary or tertiary UWB device 250 that was determined to be within association range at step 310. After sending out the association request, the primary controller may await a response from the secondary and/or tertiary UWB device 250 accepting the request before proceeding to step 312. If no acceptance is received, the primary controller may return to step 300 without performing step 312.

When algorithm 128b (FIG. 10) is modified to include steps 308, 310, and 312 of algorithm 128a, it may also be modified to include the additional step mentioned immediately above. That is, the secondary controller may proceed to send out a request to associate with another secondary or tertiary UWB device 250 that is within range and wait for its response. If a response is received, the secondary controller proceeds with the association process. If no response is received, the secondary controller does not conclude the association process and instead returns to step 322.

In yet another modification, any or all of algorithms 128a-c may be modified such that any step that involves associating a UWB device with, or disassociating a UWB device from, another device is modified to carry out a pairing process, either in lieu of, or in addition to, the association process. Thus, for example, algorithm 128a may be modified by changing step 304 to a step in which the primary controller pairs the primary UWB device 250 with the locator unit 142 instead of, or in addition to, associating the primary UWB device 250 with the locator unit 142. The rest of algorithm 128a may be modified in a similar manner. That is, at step 306, the primary controller may decide not to pair with the locator unit 142 (and/or unpair with a previously paired locator unit 142). Further, at step 312, the primary controller may pair the secondary or tertiary UWB device 250 with the primary UWB device. Steps 302 and 310 may be modified such that the primary controller determines if the other device is within a pairing range, rather than an association range. Similar modifications may be made to algorithms 128b and/or 128c.

It will also be appreciated that, in at least some embodiments, the system of the present disclosure may operate without any tertiary UWB devices 250. That is, in some embodiments, only primary and secondary UWB devices 250 may be included within the system. In still other embodiments, only primary and tertiary devices may be included. Still other combinations are possible. It will also be understood that, although FIG. 8 only includes two UWB devices 250 (temperature management device 46 and a generic UWB device 250), the number of UWB devices 250 may be varied from what is shown there. That is, the number of UWB devices 250 that may be associated with a primary or secondary UWB device 250 is not limited by the present disclosure.

In some embodiments, linked locator units 142 are configured to only allow a single primary UWB device 250 to be associated with a single linked locator unit 142. In such embodiments, unlinked locator units 142 may be configured to allow more than one primary UWB device 250 to be associated with a single unlinked locator unit 142. In some embodiments, the linked locator units 142 may be configured to act as UWB tags that send out start packets at a variable frequency (i.e. different intervals between ranging sessions) depending upon whether a primary UWB device 250 is associated with the locator unit 142 or not, and/or dependent upon other conditions (e.g. the brake status of a patient support apparatus 20 and/or AC cord status of the patient support apparatus 20). In such embodiments, the unlinked locator units 142 may be configured send out start packets at a fixed frequency, regardless of whether or not a primary UWB device 250 is associated with the locator unit 142 or not. In still other embodiments, the frequency at which the start packets are sent out by a linked or unlinked locator unit 142 may be customized by a user, such as via patient support apparatus 20 communicating a desired frequency to the locator unit 142, a server (e.g. patient support apparatus server 138) communicating the desired frequency to the locator unit 142, and/or by an electronic device 162 communicating the desired frequency to the locator unit 142.

Further details regarding the differences and similarities between linked and unlinked locator units 142 may be found in the description of the linked and unlinked wall units 60a and 60b, respectively, in commonly assigned U.S. provisional patent application Ser. No. 63/323,202 filed Mar. 24, 2022, by inventors Kirby Neihouser et al. and entitled SYSTEM FOR LOCATING PATIENT SUPPORT APPARATUSES, the complete disclosure of which is incorporated herein by reference. In some embodiments, the unlinked locator units 142 of the present disclosure may include any of the components of the unlinked wall unit 60b disclosed in the aforementioned '202 patent application. Similarly, in some embodiments, the unlinked locator units 142 of the present disclosure may omit any of the components shown in FIG. 7 herein that are not included in, or disclosed as optional in, the aforementioned '202 patent application. Still other modifications may be made to the locator units 142.

Although the decision as to whether to associate a first UWB device 250 has been described herein as being carried out by a specific controller, it will be understood that this decision may alternatively be carried out by other structures. For example, in some embodiments, controller 216 is configured to send the spatial relationship data it determines from the communications between its UWB transceiver(s) 212 and one or more other UWB devices 250 to patient support apparatus server 138 and patient support apparatus server 138 then determines whether to associate any of these devices with patient support apparatus 20. In still other embodiments, one or more of the UWB devices 250 may include its own controller that determines whether it should be associated with patient support apparatus 20 and/or with another UWB device, and that controller may then forward that information to patient support apparatus 20, to patient support apparatus server 138, to EMR server 136, and/or to another recipient, either directly or through one or more intermediaries.

It will also be understood that in any of the embodiments of the UWB devices 250 described herein, the size, shape, location, and/or other aspects of the volumes of space used to determine whether to associate two or more UWB devices 250 may be changed substantially from the two volume of space examples A and B shown in FIG. 6. For example, in some embodiments, the space volume used for determining the association with a linked locator unit 142 may be different from the space volume used to determine the association with an unlinked locator unit 142. Similarly, each different type of UWB device 250 may utilize different volumes of space that are specific to that particular type of UWB device 250. Additionally, or alternatively, individual ones of linked locator units 142 may have space volumes of different sizes and/or shapes from other individual linked locator units 142, and/or individual ones of unlinked locator units 142 may have space volumes of different sizes and/or shapes from other individual unlinked locator units 142.

In some embodiments, the controller of a UWB device 250 is configured to consult an onboard memory to determine the size, shape, and/or other information about a particular space volume to be used for determining an association status. In such embodiments, one of the controllers of the pair of UWB devices 250 uses a unique ID it receives from the other device 250 to determine the size, shape, and/or other information about the space volume to be used for associating and/or disassociating with that other device 250. In such embodiments, the UWB device's memory contains data correlating particular space volumes to particular UWB devices 250. In this manner, each UWB device 250 can potentially have different space volumes associated with it, and these definitions of these space volumes need not be transmitted to the UWB device. Instead, the onboard controller consults its onboard memory to determine the boundaries of the space volume and decide whether the spatial conditions required to associate or disassociate are met.

In some embodiments, one or more UWB devices 250 (and/or locator units 142) may be configured to use different space volumes for purposes of association than for purposes of disassociation. In other words, once a UWB device 250 has been determined to be positioned inside of a particular volume of space (and the other association conditions are met, if any), the controller of the UWB device 250 may increase the size of—and/or otherwise change one or more dimensions of—the volume of space when determining whether to disassociate the UWB device 250. In this manner, the volumes of space may have a sort of hysteresis aspect wherein a UWB device 250 has to be positioned inside of a smaller space volume in order to be associated with another UWB device 250, but thereafter can only be disassociated if it moves outside of a larger sized volume of space. In still other embodiments, the dimensions of one or more of the volumes of space are the same for both association and disassociation purposes.

The term “associates,” or its variants, as used herein, refers to the identification by a controller of a first UWB device 250 that another UWB device 250 is positioned within a sufficiently close proximity to the first UWB device 250 such that the controller of the first UWB device 250 can safely conclude that the other UWB device 250 is also intended for use with the same patient as the first UWB device 250. Alternatively, or additionally, the term “associates,” or its variants, as used herein, refers to the identification by a controller of a first UWB device 250 that another UWB device 250 is positioned within a sufficiently close proximity to the first UWB device 250 such that the controller of the first UWB device 250 can safely conclude that the location of the other UWB device 250 is the same location in the healthcare facility as the first UWB device 250. Alternatively, or additionally, the term “associates,” or its variants, as used herein, refers to the identification by a controller of a first UWB device 250 that another UWB device 250 is positioned within a sufficiently close proximity to the first UWB device 250 such that the controller of the first UWB device 250 can safely conclude that data from the first UWB device 250 and the data from the other UWB device 250 should be routed to the same destination(s) and/or via the same pathway.

In some embodiments, once an association has been made between a particular UWB device 250 and a particular patient support apparatus 20 and/or a particular locator unit 142, a further association may be made between that particular UWB device 250 and a particular patient. This task of associating and disassociating a particular patient to a particular UWB device 250 may also be carried out locally by a controller onboard the UWB device 250, or it may be carried out remotely by patient support apparatus server 138. Such remote association to a particular patient generally involves patient support apparatus server 138 using information from ADT server 170 or EMR server 136 on network 134 to determine the room location (e.g. room number and/or bed bay ID) of a particular patient, and then matching that room location with the room location of a particular UWB device 250 (which is reported to server 138 by the UWB device 250 after it, or an associated UWB device 250, has associated itself with a particular locator unit 142). In other words, server 138 consults a conventional server on network 134 that correlates specific patients to specific room numbers and/or bay areas, such as ADT server 170 and/or EMR server 136, and then uses the known room numbers and/or bay areas of specific UWB devices 250 to match a specific patient to those specific UWB devices 250.

In some embodiments, when a UWB device 250 and/or server 138 associate a UWB device 250 with a specific patient, the UWB device's controller and/or server 138 are configured to inform medical personnel (via electronic devices 162) that the UWB device 250 is associated with a specific patient. Any of the UWB devices 250 may therefore be configured to automatically forward patient temperature data, vital sign data, and/or other data from other UWB devices 250 to server 138 after the corresponding devices 250 become associated with specific patient. Such data may, in turn, be automatically forwarded by server 138 to EMR server 136 for entry into the corresponding patient's electronic medical record. Alternatively, or additionally, such data may be forwarded by server 138 to one or more electronic devices 162 associated with corresponding caregivers so that the caregivers assigned to that particular patient may be remotely informed of the data from the associated UWB device(s) 250.

Once a UWB device controller or server 138 associates a particular UWB device 250 with a particular patient, data from the associated UWB device 250 can be automatically recorded in that patient's particular electronic medical record without requiring the caregiver to associate the UWB device 250 with a particular patient and/or with the patient support apparatus 20 assigned to that patient. In other words, because the UWB devices controller automatically determines what other UWB devices 250 it is associated with, it is not necessary for a caregiver to take any manual steps to ensure that data from these UWB devices 250 is forwarded to the proper corresponding patient's electronic medical record. Further details regarding at least one manner in which this automatic patient determination may be made are found in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which is incorporated herein by reference. Additional details regarding a manner of automatically associating a UWB device with a particular patient, location, caregiver, or other parameters are found in commonly assigned Indian Patent Application number 202211062036 filed Oct. 31, 2022, in the Indian Patent Office by inventors Thomas Durlach et al. and entitled CAREGIVER ASSISTANCE SYSTEM, the complete disclosure of which is incorporated herein by reference.

In some embodiments, server 138 is configured to determine patient-to-room, patient-to-bed, patient-to-bed-bay, patient-to-caregiver, caregiver-to-room, caregiver-to-patient-support-apparatus, caregiver-to-UWB device 250, and/or caregiver-to-bed-bay correlations in any of the manners disclosed in commonly assigned U.S. patent application Ser. No. 62/826,097, filed Mar. 29, 2019 by inventors Thomas Durlach et al. and entitled PATIENT CARE SYSTEM, the complete disclosure of which is incorporated herein by reference. In some embodiments, server 138 may further be modified to carry out any of the staffing errors, and other error-notification functions, disclosed in the aforementioned '097 application.

In still other embodiments, server 138 and/or one or more UWB devices 250 may be configured to determine patient-to-room, patient-to-bed, patient-to-bed-bay, patient-to-caregiver, caregiver-to-room, caregiver-to-patient-support-apparatus, caregiver-to-UWB device 250, and/or caregiver-to-bed-bay correlations in any of the manners disclosed in commonly assigned Indian Patent Application number 202211062036 filed Oct. 31, 2022, in the Indian Patent Office by inventors Thomas Durlach et al. and entitled CAREGIVER ASSISTANCE SYSTEM, the complete disclosure of which is incorporated herein by reference. Alternatively, or additionally, the routing of data from an associated UWB device 250 may be automatically directed to one or more desired destinations (such as, but not limited to, one or more electronic devices 162) in any of the same manner disclosed in commonly assigned U.S. patent application Ser. No. 63/428,074 filed Nov. 27, 2022, by inventors Madhu Sandeep Thota et al. and entitled COMMUNICATION SYSTEM FOR PATIENT SUPPORT APPARATUSES, the complete disclosure of which is incorporated herein by reference.

It should also be noted that the routing and/or display of data from an associated UWB device 250 is carried out automatically by locator unit 142, by patient support apparatus 20, by the UWB device 250 itself, and/or by patient support apparatus server 138. That is, for example, when a user has configured a UWB device 250 to display a patient's temperature on a display device 182 (or a display coupled to an electronic device 162), it automatically forwards the temperature data to the display device after the corresponding association has been made (e.g. the temperature sensing device 250 has been associated with a display device 182, a locator unit 142 to which a display device 182 is coupled, and/or an electronic device 162 associated with the same patient and/or caregiver as the temperature sensing UWB device 250). Consequently, if a patient with a UWB device 250 that measures the patient's temperature is moved into a bay area of a patient room that includes a fixed display device 182 coupled to a locator unit 142, the temperature sensing UWB device 250 is configured to automatically start displaying the patient's temperature on that display device 182 as soon as it completes the association process with the corresponding locator unit 142, or with a patient support apparatus 20 associated with that locator unit 142, or with another UWB device 250 that is associated with that particular locator unit 142. In this manner, the caregiver sees the patient's temperature displayed on the display device 182 within seconds after moving the patient into the bay area, and the caregiver doesn't need to connect any cables, press any buttons, or take any other actions, in order for the temperature data to be displayed on display device 182. Similarly, when the patient moves out of the bay area, the display of the patient's data on the display device 182 automatically terminates.

The automatic display of data from one or more UWB devices 250 on one or more display devices 182 may be also, or alternatively, be carried out in any of the manners disclosed in commonly assigned U.S. provisional patent application 63/356,061 filed Jun. 28, 2022, by inventors Krishna Bhimavarapu et al. and entitled BADGE AND PATIENT SUPPORT APPARATUS COMMUNICATION SYSTEM, the complete disclosure of which is incorporated herein by reference.

When a patient support apparatus 20 associates itself with a particular linked locator unit 142, controller 216 selects that particular linked locator unit 142 to send the patient's voice signals to (and/or exit detection alerts to) for forwarding to nurse call system 152. It is also the linked locator unit 142 that controller 216 sends television commands to when a patient onboard patient support apparatus 20 activates one or more of the television controls 501-50r. Similarly, it is the linked locator unit 142 that controller 216 sends light commands to when a patient onboard patient support apparatus 20 activates one or more or the reading or room light controls 50s or 50t. The linked locator unit 142 that patient support apparatus 20 associates itself with is also the locator unit 142 that patient support apparatus 20 will receive audio signals from and direct to its onboard speaker(s). Such audio signals may correspond to voice signals from a remotely positioned nurse that are forwarded to the corresponding communication outlet 144 by way of nurse call system 152, or such audio signals may correspond to television audio signals that are routed from television 150 to communication outlet 144 by way of the one or more conductors 158.

In some embodiments, one or more of the UWB transceivers that are coupled to a particular UWB device 250 may be physically constructed as a tag that is attached to that particular device (as opposed to the “tag” mode of operation discussed above). Examples of the manner in which such physical tags may be constructed are disclosed in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which has already been incorporated herein by reference. The use of such physical tags allows the UWB devices 250 to automatically determine the position of tagged devices that don't have their own built-in UWB transceivers, but that instead have a UWB tag physically attached to them.

In some embodiments, one or more of the UWB transceivers disclosed herein may operate in the same manner as, and include any of the same functions as, the anchors and pseudo-anchors disclosed in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which has already been incorporated herein by reference.

In any of the embodiments disclosed herein, server 138 may be configured to additionally execute a caregiver assistance software application of the type described in the following commonly assigned patent applications: U.S. patent application Ser. No. 62/826,097, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application serial number PCT/US2020/039587 filed Jun. 25, 2020, by inventors Thomas Durlach et al. and entitled CAREGIVER ASSISTANCE SYSTEM, the complete disclosures of which are all incorporated herein by reference. That is, server 138 may be configured to share with one or more electronic devices 162 any of the information shared with the electronic devices disclosed in these aforementioned patent applications.

In some embodiments, linked locator units 142 and/or unlinked locator units 142 may include additional information stored therein that is shared with a UWB device 250 when the UWB device 250 becomes associated with the locator unit 142. Such additional information may include location information identifying the relative position of the locator unit 142 with respect to one or more other locator units 142 that are positioned nearby. Additionally or alternatively, the locator units 142 may include information regarding the thickness and/or materials of the wall to which it is attached, wherein such information provides an indication to the UWB device(s) of the amount of attenuation that UWB signals will likely experience when traveling through that wall. Additionally or alternatively, the locator units 142 may include information identifying their general location within the healthcare facility (e.g. room 400, bay A of room 302, hallway X, maintenance area Y, radiology department, emergency department, etc.) and/or information identifying a more specific location of the locator units 142 within the healthcare facility (e.g. a set of X,Y,Z coordinates in a frame of reference that includes all, or a portion of, the healthcare facility; a height on the wall 154, a distance from one or more landmarks and/or architectural features within the healthcare facility, and/or other more specific information). In some embodiments, the UWB device 250 is adapted to utilize this information to determine its location within the healthcare facility and/or to determine whether it is positioned on the same side of a wall as a particular locator unit 142. In some embodiments, one or more UWB devices 250 and/or locator units 142 include any of the same structures, functions, and/or features of any of the patient support apparatuses and/or wall units disclosed in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosure of which has already been incorporated herein by reference.

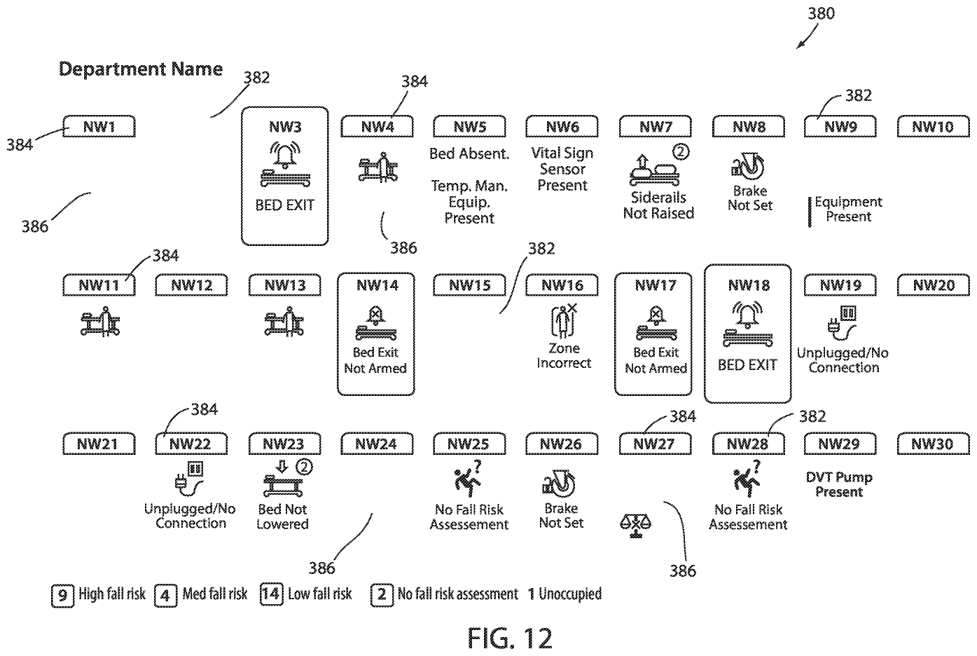

FIG. 12 illustrates one example of a dashboard screen 380 that may be displayed on a display of an electronic device 162, particularly an electronic device 162 having a relatively large display screen size, such as a television or computer monitor. Dashboard screen 380 includes a plurality of room icons 382 (i.e. enclosures that are defined by rectangles having rounded corners). Each room icon 382 corresponds to a particular room and/or bay within an actual room of the healthcare facility in which the system of the present disclosure is installed. Thus, in the example shown in FIG. 12, there are thirty room icons 382. Each room icon 382 includes a header portion 384 that identifies the particular room in the healthcare facility to which the room icon 382 corresponds and a body portion 386 that, as will be discussed more below, may display information about the status of one or more UWB devices 250 positioned within that particular room.

The content of screen 380 is controlled by a software application executed on patient support apparatus server 138. That is, server 138 forward the content of screen 380 to one or mor appropriate electronic devices 162 and instructs the electronic devices 162 to display the contents of screen 380. In general, the software application instructs the electronic devices to display data regarding any relevant aspects of one or more UWB devices 250 that are positioned within the corresponding room. For example, in the example of FIG. 12, room NW5 is missing a patient support apparatus 20, but does have a temperature management device 46 present therein. Room NW6 has a vital sign sensor present in that room. Room NW29 has a DVT pump present. In some embodiments, server 138 may be configured to display only a generic indicator that equipment is present in the room, such as the generic indicator “equipment present” shown for room NW9. If the electronic device 162 is a touch screen, server 138 may be configured to display more information about the equipment in the room in response to a user pressing on the generic “equipment present” indicator.

In some embodiments, server 138 may display the screen 380 on one or more electronic devices 162 in any of the same manners, and/or with any of the same functionality as, the software application 100 that is adapted to be executed on the patient support apparatus server 86 disclosed in commonly assigned Indian Patent Application number 202211062036 filed Oct. 31, 2022, in the Indian Patent Office by inventors Thomas Durlach et al. and entitled CAREGIVER ASSISTANCE SYSTEM, the complete disclosure of which is incorporated herein by reference.

In some embodiments, it will be understood that the ranging information exchanged between UWB devices 250 (including locator unit 142) may be used to not only determine the distance between the UWB devices 250, but also angular orientation of the devices with respect to each other. In some embodiments, this ranging information may be of the same type, and/or processed in the same manner, as the ranging information discussed in, and illustrated in FIGS. 16 and 17 of, commonly assigned PCT patent application serial number PCT/US2022/017616 filed Feb. 24, 2022, by applicant Stryker Corporation and entitled SYSTEM FOR DETERMINING PATIENT SUPPORT APPARATUS AND MEDICAL DEVICE LOCATION, the complete disclosure of which is incorporated herein by reference.

It will be understood by those skilled in the art that the use of the term “transceiver” throughout this specification is not intended to be limited to devices in which a transmitter and receiver are necessarily within the same housing, or share some circuitry. Instead, the term “transceiver” is used broadly herein to refer to both structures in which circuitry is shared between the transmitter and receiver, and transmitter-receivers in which the transmitter and receiver do not share circuitry and/or a common housing. Thus, the term “transceiver” refers to any device having a transmitter component and a receiver component, regardless of whether the two components are a common entity, separate entities, or have some overlap in their structures.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A device comprising:
an ultra-wideband transceiver; and
a controller in communication with the ultra-wideband transceiver, the controller adapted to use the ultra-wideband transceiver to attempt to determine a first distance between the ultra-wideband transceiver and a patient support apparatus; wherein, if the controller is unable to determine the first distance between the ultra-wideband transceiver and the patient support apparatus, or if the first distance is greater than a first threshold, the controller is further adapted to use the ultra-wideband transceiver to attempt to determine a second distance between the ultra-wideband transceiver and a fixed locator mounted at a fixed location within a healthcare facility.

2. The device of claim 1 wherein the controller is further adapted to determine if the second distance is less than a second threshold, and if the second distance is less than the second threshold, to associate the device with the fixed locator.

3. The device of claim 2 further comprising a network transceiver adapted to communicate with a network of the healthcare facility, wherein the controller is further adapted to send a fixed locator ID to the network if the second distance is less than the second threshold, and to not send the fixed locator ID to the network if the second distance is greater than the second threshold.

4. The device of claim 1 wherein the controller and the ultra-wideband transceiver are adapted to act as a UWB tag when the controller is unable to determine the first distance or when the first distance is greater than the first threshold, and wherein the controller and the ultra-wideband transceiver are further adapted to act as a UWB anchor when the first distance is less than the first threshold.

5. The device of claim 4 wherein, when the controller and the ultra-wideband transceiver are acting as the UWB tag, the ultra-wideband transceiver is adapted to periodically transmit a discovery packet to any other ultra-wideband transceivers within range and to wait for a response from the any other ultra-wideband transceivers; and when the controller and the ultra-wideband transceiver are acting as the UWB anchor, the controller and the ultra-wideband transceiver are adapted to respond to a discovery packet transmitted by another ultra-wideband transceiver.

6. (canceled)

7. The device of claim 5 wherein, when the controller and the ultra-wideband transceiver are acting as the UWB tag, the ultra-wideband transceiver and controller are adapted to ignore a discovery packet transmitted by another ultra-wideband transceiver; and when the controller and the ultra-wideband transceiver are acting as the UWB anchor, the ultra-wideband transceiver is adapted to not transmit any discovery packets to another ultra-wideband transceiver.

8. (canceled)

9. The device of claim 1 wherein the device is a thermal control unit adapted to control a patient's temperature during a thermal therapy session; and the thermal control unit includes:
a first fluid port adapted to fluidly couple to a first hose;
a second fluid port adapted to fluidly couple to a second hose;
a fluid channel fluidly coupling the first fluid port to the second fluid port;
a pump for pumping fluid through the thermal control unit;
a heat exchanger adapted to add or remove heat from the fluid;
a fluid temperature sensor adapted to sense a temperature of the fluid;
a patient temperature sensor port adapted to receive patient temperature readings from a patient temperature sensor; and
a temperature controller adapted to control the heat exchanger in order to control the patient's temperature.

10. (canceled)

11. The device of claim 1 wherein the controller is further adapted to receive a fixed locator ID from the fixed locator if the second distance is less than a second threshold, and wherein the controller is further adapted to transmit the fixed locator ID using the ultra-wideband transceiver to another device if the another device is within a third threshold.

12-18. (canceled)

19. A device comprising:
an ultra-wideband transceiver; and
a controller in communication with the ultra-wideband transceiver, the controller adapted to use the ultra-wideband transceiver to automatically attempt to pair with a patient support apparatus; wherein, if the controller is unable to pair with the patient support apparatus, the controller is further adapted to use the ultra-wideband transceiver to automatically attempt to pair with a fixed locator mounted at a fixed location within a healthcare facility.

20. The device of claim 19 wherein the controller is further adapted to not attempt to pair with the fixed locator if the controller is able to pair with the patient support apparatus.

21. The device of claim 19 wherein the controller is further adapted, after pairing with the fixed locator, to automatically re-attempt to pair with the patient support apparatus and to automatically unpair the device from the fixed locator if the controller is able to pair with the patient support apparatus.

22. (canceled)

23. The device of claim 19 wherein the controller is adapted to receive a fixed locator ID from the fixed locator when the device is paired with the fixed locator, and the controller is further adapted to send the fixed locator ID to a network using a network transceiver adapted to communicate with the network.

24. (canceled)

25. The device of claim 23 wherein the controller is further adapted to send the fixed locator ID to another device if the another device is within a threshold distance of the device.

26. The device of claim 19 wherein the controller is configured to attempt to pair with the patient support apparatus by determining a first distance between the ultra-wideband transceiver and a second ultra-wideband transceiver on the patient support apparatus; and the controller is configured to attempt to pair with the fixed locator by determining a second distance between the ultra-wideband transceiver and a third ultra-wideband transceiver on the fixed locator.

27-38. (canceled)

39. A system comprising:
a server adapted to be accessible from a network of a healthcare facility; and
a device including a first UWB transceiver, a controller, and a network transceiver adapted to be able to communicate with the server;
wherein the server is adapted to equate a location of the device with a location of a patient support apparatus if the device is positioned within a first distance of the patient support apparatus, and wherein the server is adapted to equate the location of the device with a location of a fixed locator if both of the following are true: (1) the device is not positioned within the first distance of the patient support apparatus, and (2) the device is positioned within a second distance of the fixed locator.

40. The system of claim 39 wherein the server is adapted to receive a fixed locator ID from the device if the device is not positioned within the first distance of the patient support apparatus and the device is positioned within the second distance of the fixed locator; and to receive the fixed locator ID from the patient support apparatus if the device is positioned within the first distance of the patient support apparatus.

41. (canceled)

42. The system of claim 39 wherein the fixed locator is adapted to transmit a fixed locator ID to the patient support apparatus if the patient support apparatus if positioned within a third distance of the fixed locator, to not transmit the fixed locator ID to the patient support apparatus if the patient support apparatus is positioned outside of the third distance from the fixed locator, to transmit the fixed locator ID to the device if the device is positioned within the second distance of the fixed locator, and to not transmit the fixed locator ID to the device if the device is positioned outside of the second distance from the fixed locator.

43. The system of claim 39 wherein the server is adapted to receive data from the device after the data passes through the patient support apparatus if the device is positioned within the first distance of the patient support apparatus, and to receive data from the device without the data passing through the patient support apparatus if both of the following are true: (1) the device is not positioned within the first distance of the patient support apparatus, and (2) the device is positioned within the second distance of the fixed locator.

44. The system of claim 39 wherein the server is in communication with a display, and the server is adapted to instruct the display to display the location of the device in the same manner whether the device is positioned within the first distance of the patient support apparatus or the device is positioned within the second distance of the fixed locator.

45-61. (canceled)

62. The system of claim 39 wherein the controller is adapted to use radio frequency (RF) communication between the first UWB transceiver and a badge worn by a user to determine a position of the badge relative to the device; to receive a badge identifier from the badge; to determine if the badge is positioned inside or outside of a threshold distance; and to transmit the badge identifier to the server using the network transceiver if the badge is inside the threshold distance.

63. (canceled)
